(19) United States
(12) Patent Application Publication
(10) Pub. No.: US 2014/0179544 A1
(43) Pub. Date: Jun. 26, 2014

(54) COMPOSITIONS, SYSTEMS AND METHODS FOR DROPLET FORMATION, SPACING AND DETECTION

(71) Applicant: Bio-Rad Laboratories, Inc., Hercules, CA (US)

(72) Inventors: Erin Steenblock, Hercules, CA (US); Amy L. Hiddessen, Hercules, CA (US); Ben Hindson, Hercules, CA (US); Kevin Ness, Hercules, CA (US)

(73) Assignee: Bio-Rad Laboratories, Inc., Hercules, CA (US)

(21) Appl. No.: 14/020,742

(22) Filed: Sep. 6, 2013

Related U.S. Application Data
(60) Provisional application No. 61/697,982, filed on Sep. 7, 2012.

(51) Int. Cl. C12Q 1/68

(52) U.S. Cl. CPC C12Q 1/686
USPC 506/9; 506/36

(57) ABSTRACT

The present disclosure provides assays and devices for forming, spacing, and/or detecting droplets. The droplets may be emulsions composed of two or more immiscible fluids. An emulsion can be a double emulsion, such as water-in-oil droplets that are present in a continuous aqueous phase. The double emulsion can be formed when the water-in-oil droplets are contacted with one or more streams of aqueous fluid(s). This disclosure also provides a variety of additives that can be added to the fluids.

### CROSS-REFERENCE

This application claims the benefit of U.S. Provisional Application No. 61/697,982, filed Sep. 7, 2012, which application is incorporated herein by reference in its entirety.

### BACKGROUND

Assays for determining the presence, quantity, activity, and/or other properties or characteristics of components in a sample play a valuable role in many diverse biological and clinical applications. In some cases, the components of interest within a sample—e.g., a nucleic acid, an enzyme, a virus, a bacterium—are only minor constituents of the sample and may, therefore, be difficult to detect or quantitate.

Certain biological assays, such as the polymerase chain reaction (PCR) assay, can be quantitative in specific settings. For example, real-time PCR (which generally involves monitoring the progression of amplification using fluorescence probes) can permit quantification of target nucleic acids in a sample, particularly where the target nucleic acids are somewhat abundant.

Digital PCR is also a quantitative PCR assay. In digital PCR, a sample containing PCR reagents and target nucleic acid molecules is distributed across multiple partitions, such that each individual partition contains on average one or fewer target nucleic acid molecules. After amplification, reactions containing one or more templates are generally detectable and can emit a signal such as a fluorescent signal. Droplet digital PCR is a form of digital PCR that uses fluidic droplets for the partitions. The steps for droplet digital PCR generally involve (1) partitioning a fluid sample containing PCR reagents and nucleic acid target molecule(s) into multiple droplets, (2) performing an amplification cycle on the droplets, and (3) detecting the presence of nucleic acids in the droplets. A nucleic acid sample can be partitioned into multiple droplets using oil and emulsion chemistry. For example, an aqueous sample can be partitioned into multiple emulsified droplets in a continuous oil phase using microfluidics technologies.

### SUMMARY

The present disclosure provides compositions, systems and methods that may be employed for use in droplet detection. Compositions, systems and methods of the present disclosure can enable improved droplet detection in cases in which, for example, an aqueous fluid is used as the carrier fluid.

In some examples, droplets comprising samples to be detected are generated and directed through a fluid flow path in sensing communication with a droplet detector. The droplets are directed through the fluid flow path using an oil-immiscible or aqueous carrier fluid. In some situations, the droplets are directed along the fluid flow path through a virtual capillary.

In an aspect, the present disclosure provides a system, device or kit for detecting droplets, comprising: (a) a detector device comprising an input flow path, an intersection region, and an output flow path, wherein the intersection region is downstream of the input flow path and the output flow path is downstream of the intersection region; (b) droplets located within the input flow path; and (c) an aqueous fluid for separating the droplets wherein the droplets are introduced to the aqueous fluid at the intersection region. The input flow path may comprise a continuous phase of non-aqueous fluid. In some embodiments, the non-aqueous fluid is an aqueous-immiscible fluid. In a further embodiment, the non-aqueous fluid is an oil. The output flow path may comprise a continuous phase of aqueous fluid. In some embodiments, the aqueous fluid comprises a surfactant. The droplets in the output flow path may have an inner core containing an aqueous fluid that is encapsulated with a non-aqueous fluid. In some embodiments, the non-aqueous fluid is a continuous phase. In some other embodiments, the non-aqueous fluid is a discontinuous phase. Alternatively, the output flow path may comprise a continuous phase of non-aqueous fluid. In some embodiments, the inner wall of the output flow path is covered by the aqueous fluid. In some embodiments, emulsified droplets flow out of the output flow path in a stream which has a diameter substantially smaller than the diameter of the output flow path. In another aspect, the present disclosure provides a system for detecting droplets, comprising: (a) a detector device comprising an input flow path, an intersection region, and an output flow path, wherein the intersection region is downstream of the input flow path and said output flow path is downstream of said intersection region; and (b) an oil-immiscible fluid for separating said droplets, wherein said oil-immiscible fluid is introduced to said droplets at said intersection region. In some case, the continuous phase of fluid within the input flow path is a non-aqueous fluid and the inner surface of the output flow path is coated with the oil-immiscible fluid.

Additionally, the present disclosure provides methods for separating droplets. In an aspect, the present disclosure provides a method of separating droplets, comprising: (a) flowing a stream of non-aqueous fluid comprising said droplets along a flow path comprising: (i) an input flow path, (ii) an intersection region, and (iii) a downstream output flow path; and (b) introducing a stream of oil-immiscible fluid to said intersection region; wherein the average distance between said droplets in said output flow path is greater than the average distance between said droplets within said input flow path. In another aspect, the present disclosure provides a method of separating droplets, comprising: (a) flowing a stream of non-aqueous fluid comprising the droplets along a flow path comprising: (i) an intersection region and (ii) a downstream output flow path; and (b) introducing a stream of oil-immiscible fluid to said intersection region; wherein said droplets are heated prior to entering said intersection region. In yet another aspect, the present disclosure provides a method of detecting droplets, comprising: (a) flowing a stream of non-aqueous fluid through a continuous flow path comprising an intersection region and a downstream detection region, wherein said non-aqueous fluid comprises said droplets; (b) introducing a stream of oil-immiscible fluid to said intersection region; and (c) detecting a signal from the droplets as they pass through said downstream detection region. The output flow path may comprise: (a) a continuous phase of oil-immiscible fluid; and (b) aqueous droplets encapsulated by a layer of non-aqueous fluid. Additionally, the flow paths of the non-aqueous fluid and that of the oil-immiscible fluid may have different angles, ranging from 1 degree to 90 degree inclusive. In one embodiment, the two flow paths are substantially perpendicular.

The oil-immiscible fluid can comprise a gas or mixture of gases, such as air. Alternatively, the oil-immiscible fluid can comprise water. When the oil-immiscible fluid comprises water, the water may comprise at least one additive. The at least one additive may adjust properties of water, for example, surface tension, viscosity, tendency to foam and anti-bacteria or anti-microbial activity. Example of additions may include, but are not limited to, surfactant, glycerol, antimicrobial agent and antifoaming agent. Any of these above mentioned agents can be uses alone or in combination. In some case, the oil-immiscible fluid comprises at least one surfactant and glycerol. In some other cases, the oil-immiscible fluid comprises at least one surfactant, at least one antimicrobial agent and glycerol.

The surfactant can be ionic or non-ionic. In some cases, the surfactant is a block copolymer of polypropylene oxide and polyethylene oxide. In some cases, the surfactant is a fluorinated surfactant. The fluorinated surfactant may be negatively charged or may comprise a carboxylate group. The amount of surfactant used may depend on the desired properties of the fluid. The weight of the surfactant may be at least 0.001%, at least 0.01%, at least 0.1%, at least 1%, at least 5% or even more of the weight of the fluid they are added to. In some cases, the amount of surfactant is about 1%, about 2%, about 3%, about 4%, about 5%, about 6%, about 7%, about 8%, about 9%, about 10%, about 11%, about 12%, about 13%, about 14%, about 15% or about 20%. In some cases, the amount of surfactant is in a range of 0.1%-99% about 1%-99%, 3%-99%, 4%-99%, 5%-99%, 10%-99%, 1%-20%, 1%-30% or 1%-40 the weight of the fluid they are added to.

The non-aqueous fluid can comprise an oil selected from the group consisting of a silicone oil, a mineral oil, a hydrocarbon oil, a fluorocarbon oil, a vegetable and a soybean oil. In some embodiments, the non-aqueous fluid comprises a surfactant. The droplets may be aqueous droplets encapsulated by the non-aqueous fluid. Upon flowing to the intersection region, the droplets may be further emulsified. The flowing can be achieved with under negative or positive fluidic pressure. In some embodiments, the flowing is achieved with at least one syringe pump.

The present disclosure enables detection of droplets with different sizes and properties. In some cases, the droplets have varying sizes. In some cases, the droplets are emulsified droplets. The droplets may comprise a nucleic acid or a product of a nucleic acid amplification reaction. In further embodiments, each of the droplets, on average, comprises less than five target nucleic acids.

The use of a non-aqueous fluid and an oil-immiscible fluid can create a virtual capillary in the output flow path. The inner wall of the output flow path may be coated with the oil-immiscible fluid, thus reducing aperture of the output flow path. The thickness of the coating layer may be at least 0.01%, at least 0.1%, at least 1%, at least 5%, at least 10%, at least 15%, at least 20%, at least 25%, at least 30%, at least 35%, at least 40%, at least 50%, at least 60%, at least 70%, at least 80%, at least 90% or even more of the diameter of the output flow path. In some cases, the thickness may be in a range of 1%-90%, 5%-90%, 10%-90%, 15%-90%, 20%-90%, 25%-90%, 30%-90%, 40%-90%, 50%-90%, 5%-95%, 10%-95%, 15%-95%, 30%-95% or 50%-95% of the diameter of the output flow path. The formation of a virtual capillary may allow the droplets flowing through the output flow path serially and substantially centered, regardless of their sizes.

In an aspect, a system for detecting droplets comprises (a) a detector device comprising an input flow path, an intersection region, and an output flow path, wherein the intersection region is downstream of the input flow path and the output flow path is downstream of the intersection region; (b) droplets located within the input flow path; and (c) an aqueous fluid for separating the droplets, wherein the droplets are introduced to the aqueous fluid at the intersection region. In an embodiment, the input flow path comprises a continuous phase of non-aqueous fluid. In another embodiment, the non-aqueous fluid is an aqueous-immiscible fluid. In another embodiment, the non-aqueous fluid is an oil. In another embodiment, the output flow path comprises a continuous phase of aqueous fluid. In another embodiment, the aqueous fluid comprises a surfactant. In another embodiment, the droplets in the output flow path each have an inner core containing an aqueous fluid that is encapsulated with a non-aqueous fluid. In another embodiment, the non-aqueous fluid is a continuous phase. In another embodiment, the non-aqueous fluid is a discontinuous phase.

In an embodiment, the aqueous fluid separates the droplets sequentially. In another embodiment, the system further comprises a detector in sensing communication with at least a portion of the output flow path, wherein the detector is configured to detect the presence or absence of an individual droplet among the droplets. In another embodiment, the detector is in optical communication with at least a portion of the output flow path.

In another aspect, a system for droplet detection comprises (a) a detector device comprising an input flow path, an intersection region downstream of the input flow path, and an output flow path downstream of the intersection region, wherein the input flow path comprises a fluid with a continuous phase that is a non-aqueous fluid, and wherein an inner surface of the output flow path is coated with the non-aqueous fluid with a thickness that is at least about 0.01% of a diameter of the output flow path, which thickness narrows an aperture of the output flow path; (b) droplets located within the input flow path and the output flow path, wherein the droplets in the output flow path each has an inner core containing an aqueous fluid that is encapsulated with the non-aqueous fluid; and (c) an oil-immiscible fluid that separates the droplets, wherein the oil-immiscible fluid is introduced to the droplets at the intersection region.

In an embodiment, the thickness is at least about 0.1% of the diameter of the output flow path. In another embodiment, the thickness is at least about 1% of the diameter of the output flow path. In another embodiment, the thickness is at least about 5% of the diameter of the output flow path. In another embodiment, the thickness is in a range of about 1%-90% of the diameter of the output flow path. In another embodiment, the system further comprises droplets in the output flow path, wherein the droplets are serially and substantially centered.

Systems above or elsewhere herein, alone or in combination, can comprise droplets each comprising a nucleic acid. The nucleic acid can be a nucleic acid sample or a partition thereof. In some embodiments, the droplets can comprise a product of a nucleic acid amplification reaction. In some embodiments, the droplets are emulsified droplets. In some embodiments, each of the droplets comprises, on average, less than five target nucleic acids.

Systems above or elsewhere herein, alone or in combination, can comprises an oil-immiscible fluid comprising a surfactant. In some embodiments, the surfactant is an ionic surfactant. In some embodiments, the surfactant is a non-ionic surfactant. In some embodiments, the surfactant is greater than about 0.01% of the weight of the total aqueous fluid. In some embodiments, the surfactant is greater than about 0.1% of the weight of the total aqueous fluid. In some embodiments, the surfactant is greater than 0.5% of the weight of the total aqueous fluid. In some embodiments, the surfactant is in a range of 0.5% to 95.0% of the weight of the total aqueous fluid, inclusive.

Systems above or elsewhere herein, alone or in combination, can comprise a detector in sensing communication with at least a portion of the output flow path. The detector can be configured to detect the presence or absence of an individual droplet among the droplets. In some embodiments, the detector is in optical communication with at least a portion of the output flow path.

In another aspect, a method for separating and/or detecting droplets comprises (a) flowing a stream of a non-aqueous fluid comprising droplets along a flow path comprising (i) an input flow path, (ii) an intersection region downstream of, and in fluid communication with, the input flow path, and (iii) an output flow path downstream of, and in fluid communication with, the intersection region; and (b) introducing a stream of oil-immiscible fluid to the intersection region to form a stream comprising the droplets in the output flow path, wherein the average distance between the droplets in the output flow path is greater than the average distance between the droplets within the input flow path. In an embodiment, the droplets flow through the output flow path serially and substantially centered. In another embodiment, the droplets comprise a nucleic acid. In another embodiment, the droplets comprise a product of a nucleic acid amplification reaction. In another embodiment, the method further comprises detecting the presence or absence of the droplets using a detector operably coupled to at least a portion of the output flow path. In another embodiment, the average distance between the droplets in the output flow path is at least 1.2 times the average distance between the droplets in the input flow path.

In another aspect, a method for separating and/or detecting droplets comprises (a) flowing a stream of a non-aqueous fluid along a flow path comprising (i) an input flow path, (ii) an intersection region downstream of, and in fluid communication with, the input flow path, and (iii) an output flow path downstream of, and in fluid communication with, the intersection region, wherein the stream of non-aqueous fluid comprises droplets that are heated prior to entering the intersection region; and (b) introducing a stream of oil-immiscible fluid to the intersection region to form a stream comprising the droplets in the output flow path. In an embodiment, the droplets flow through the output flow path serially and substantially centered. In another embodiment, the droplets comprise a nucleic acid. In another embodiment, the droplets comprise a product of a nucleic acid amplification reaction. In another embodiment, the method further comprises detecting the presence or absence of the droplets using a detector operably coupled to at least a portion of the output flow path.

In another aspect, a method for detecting droplets comprises (a) flowing a stream of non-aqueous fluid through a continuous flow path comprising an intersection region and a downstream detection region, wherein the non-aqueous fluid comprises droplets; (b) introducing a stream of oil-immiscible fluid to the intersection region; and (c) detecting, with the aid of a detector operably coupled to at least a portion of the detection region, a signal from the droplets upon flow of the droplets through the downstream detection region.

In some embodiments, the output flow path comprises a continuous phase of oil-immiscible fluid; and aqueous droplets encapsulated by a layer of non-aqueous fluid. In some embodiments, flow paths of the non-aqueous fluid and flow paths of the oil-immiscible fluid are substantially perpendicular to one another. In some embodiments, the oil-immiscible fluid comprises air. In some embodiments, the oil-immiscible fluid comprises water.

In some embodiments, the oil-immiscible fluid further comprises a surfactant. In some cases, the weight of the surfactant is at least about 0.001%, at least about 0.01%, at least about 0.1%, or at least about 1% of the weight of the water. In some embodiments, the weight of the surfactant is in a range of about 0.1%-99% of the weight of the water.

In some embodiments, the oil-immiscible fluid further comprises glycerol. In some cases, the weight of the glycerol is at least about 0.01% or at least about 0.1% of the weight of the water. In some situations, the weight of the glycerol is in a range of about 0.1% of the weight of the water.

In some embodiments, the oil-immiscible fluid further comprises an antimicrobial agent. In some embodiments, the oil-immiscible fluid further comprises an antifoaming agent.

In some embodiments, the non-aqueous fluid comprises an oil selected from the group consisting of a silicone oil, a mineral oil, a hydrocarbon oil, a fluorocarbon oil, a vegetable and a soybean oil. In some cases, the oil comprises a surfactant. In some embodiments, the surfactant is selected from the group consisting of a fluorocarbon, a hydrocarbon or a silicone surfactant. In some examples, the surfactant comprises a fluorinated surfactant. The fluorinated surfactant can be negatively charged. The fluorinated surfactant can comprise a carboxylate group.

In some embodiments, the droplets comprise aqueous droplets encapsulated by the non-aqueous fluid. An aqueous phase of the droplets can comprise a surfactant. In some cases, the surfactant is an ionic surfactant. As an alternative, the surfactant can be a non-ionic surfactant. In some examples, the surfactant is a block copolymer of polypropylene oxide and polyethylene oxide.

The droplets can have varying sizes. In some embodiments, the average distance between the droplets in the output flow path is at least 1.2 times the average distance between the droplets in the input flow path. In some embodiments, the droplets are substantially centered within the output flow path. The droplets can be emulsified droplets.

In some embodiments, flowing the droplets comprises operating one or more syringe pumps. The syringe pumps can be configured to induce the flow of a fluid comprising the droplets through a fluid flow path.

In some embodiments, the oil-immiscible fluid forms a virtual capillary within the output flow path. In some examples, the virtual capillary is a capillary or channel that is defined by an outer fluid layer.

In some embodiments, at least one, some, or all of the droplets comprise a nucleic acid or a portion (or partition) thereof. In some situations, the droplets comprise a product of a nucleic acid amplification reaction.

In some embodiments, each of the droplets comprises, on average, less than five target nucleic acids. Alternatively, each of the droplets can comprise, on average, from one to five target nucleic acids.

In some embodiments, the oil-immiscible fluid comprises at least one surfactant and glycerol. The oil-immiscible fluid can comprise at least one surfactant, at least one antimicrobial agent and glycerol. In some cases, the droplets flow through the detection region serially and substantially centered. The average distance between the droplets in the detection region may be at least about 1.2 times the average distance between the droplets in an input region upstream of the intersection region.

Another aspect provides a computer readable medium comprising machine-executable code that, upon execution by a computer processor, implements any of the methods or elsewhere herein, alone or in combination.

Another aspect provides a system comprising a computer processor and a memory location comprising machine-executable code that, upon execution by the computer processor, implements any of the methods or elsewhere herein, alone or in combination.

In another aspect, a system for detecting droplets comprises (a) a detector device comprising: (i) a flow path comprising an input flow path, an intersection region downstream of the input flow path, and an output flow path downstream of the intersection region; and (ii) a detector operably coupled to the output flow path; and (b) a computer processor operably coupled to the detector device, wherein the computer processor is programmed to: (i) flow a stream of a non-aqueous fluid comprising droplets from the input flow path to the intersection region; (ii) introduce a stream of oil-immiscible fluid to the intersection region to form a stream comprising the droplets in the output flow path; and (iii) regulate the detection of the droplets in the output flow path with the aid of the detector. In an embodiment, the computer processor is programmed to regulate fluid flow such that the average distance between the droplets in the output flow path is at least about 1.2 times the average distance between the droplets in the input flow path. In another embodiment, the computer processor is programmed to flow the droplets through the output flow path serially and substantially centered. In another embodiment, the system further comprises a pump for directing fluid flow through the flow path. In another embodiment, the computer processor is programmed to regulate the operation of the pump to flow the stream of the non-aqueous fluid and/or introduce the stream of oil-immiscible fluid to the intersection region.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG.” and “Figure” herein), of which:

FIG. 1 illustrates a general workflow for droplet digital PCR (ddPCR) technology.

FIG. 2 illustrates an exemplary flowchart depicting the steps of a fluorescence detection method in a flow-based system.

FIG. 3 illustrates an exemplary device for spacing and detecting droplets in a flow system.

FIG. 4 illustrates another exemplary device for spacing and detecting droplets in a flow system.

FIG. 5 is a graphical representation of the fluorescence amplitudes of droplets detected after the droplets are contacted with an oil-immiscible fluid comprising water.

FIG. 6 is a graphical representation of the fluorescence amplitudes of droplets detected after the droplets are contacted with an oil-immiscible fluid comprising water and 8% glycerol.

FIG. 7 is a graphical representation of the fluorescence amplitudes of droplets detected after the droplets are contacted with an oil-immiscible fluid comprising water and 16% glycerol.

FIG. 8 is a graphical representation of the fluorescence amplitudes of droplets detected after the droplets are contacted with an oil-immiscible fluid comprising water and 1% Pluronic® surfactant (upper panel, FIG. 8A) or with an oil-immiscible fluid comprising water, 8% glycerol, and 2% Pluronic® surfactant (lower panel, FIG. 8B).

FIG. 9 is a graphical representation of the fluorescence amplitudes of droplets detected after the droplets are contacted with an oil-immiscible fluid comprising water (upper panel) or with a focusing fluid comprising an oil (lower panel).

FIG. 10 is a graphical representation of the fluorescence amplitudes of droplets detected after the droplets are flowed through a detector device using a 10:1 singulation ratio.

FIG. 11 is a graphical representation of the fluorescence amplitudes of droplets detected after the droplets are contacted with an oil-immiscible fluid comprising water, 8% glycerol, and 2% Pluronic® F-68 surfactant.

FIG. 12 is a graphical representation of the fluorescence amplitudes of droplets detected after the droplets are contacted with a focusing fluid comprising HFE-7500 oil.

FIG. 13 is a graphical representation of detected signal after the droplets are contacted with a focusing fluid and either the tip is not wiped (left panel) or the tip is wiped (right panel).

FIG. 14 shows a computer system that is programmed or otherwise configured to implement methods of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed in practicing the invention.

The term “channel,” as used herein, generally refers to a fluid flow path for conveying matter (e.g., a fluid) from one point to another.

The term “virtual capillary,” as used herein, generally refers to a capillary or channel that is defined by one or more outer fluid (e.g., liquid) layers. An outer fluid layer can be adjacent to (e.g., directly adjacent to and in contact with) a wall of a physical capillary or channel. In some examples, a virtual capillary is a fluid channel that is defined or otherwise characterized by an outer fluid layer. An example of a virtual capillary is a double emulsion.

In some examples, in a virtual capillary, the outer fluid can flow along the outer wall of a physical capillary. The outer fluid can flow substantially within a hollow cylinder. The outer wall of the hollow cylinder can be defined by the physical capillary. The inner wall of the hollow cylinder can define an inner core through which an inner fluid can flow. The inner fluid can be immiscible with the outer fluid. The radial location of the inner wall of the hollow cylinder can vary with time, axial location within the physical capillary, and flow conditions; that is, the hollow cylinder can be said to have a “soft” inner wall. The inner fluid can be an emulsion. The emulsion may comprise a discontinuous phase and an immiscible continuous phase.

The terms “downstream” and “upstream,” as used herein, generally refer to the position of a species, such as one or more droplets, along a system or device(s), such as along a fluid flow path in a droplet generator. Such terms can refer to the relative position of species. For example, a first droplet downstream of a second droplet can be further along a fluid flow path than the second droplet—the second droplet, in such a case, can be upstream of the first droplet. The first droplet can be in the same device as the second droplet or a separate device. The first and second droplets can be in separate devices. The devices may or may not be connected, such as by a flow path.

The term “emulsion,” as used herein, generally refers to a mixture of two or more fluids that are normally immiscible. An emulsion can include a first phase in a second phase, such as an aqueous phase in an oil phase or vice versa. The first phase can be a discontinuous (or dispersed) phase and the second phase can be a continuous phase. In some cases, an emulsion includes more than two phases. An emulsion can include multiple emulsions. An emulsion can include a droplet in another droplet, which other droplet, in some cases, is in another droplet. In some examples, an emulsion is a double emulsion, triple emulsion, or quadruple emulsion.

The present disclosure provides methods, devices, compositions, kits, and systems for separating and detecting emulsified droplets, generally within a detector device. The detector device can comprise an input flow path (e.g., channel, tube, capillary, etc.) connected to at least one intersection region that is connected to an output flow path. The droplets can flow through the input flow path within a particular fluid, in some cases as an emulsion. At or near the intersection region, a fluid that is immiscible with that particular fluid can be introduced to the droplet or droplet emulsion. The immiscible fluid may be delivered through at least one delivery flow path to the intersection region. The emulsified droplets in the output flow path generally flow to at least one downstream detection region. In some cases, the detector device comprises a detector that detects a signal emitted from the emulsified droplets; such detection may occur in a detection region. Examples of droplet detectors are provided in U.S. Patent Publication No. 2010/0173394 to Colston et al. (“Droplet-based assay system”), which is entirely incorporated herein by reference for all purposes.

The methods and devices provided herein may enable modulation of the spacing between droplets. For example, the device may increase the spacing between droplets in the output flow path. This increase in spacing may occur as a result of the introduction of an immiscible fluid at the intersection region. In some cases, the average distance between the droplets in the output flow path may be greater than the average spacing between the droplets in the input flow path. In some cases, the device may be able to decrease, or otherwise modulate, the spacing between the droplets.

The fluids used in the devices described herein may be oil-immiscible (e.g., aqueous, air, etc.) or non-aqueous, or a combination of both. In some embodiments, the non-aqueous fluid is an oil. The oil can be selected from the group consisting of a silicone oil, a mineral oil, a hydrocarbon, a fluorocarbon oil, a vegetable and a soybean oil. The aqueous fluid may be any appropriate aqueous fluid including water.

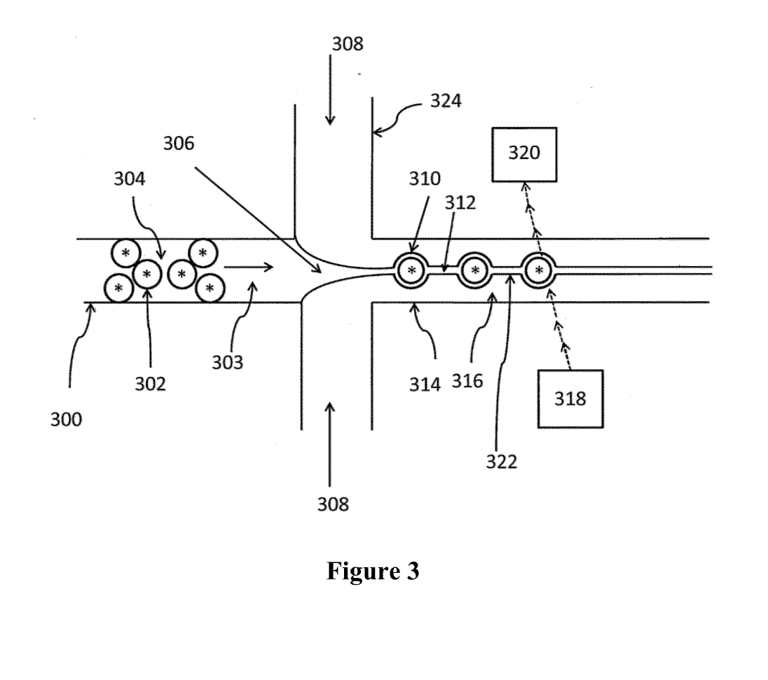

The immiscible fluid that is introduced to the droplets at or near the intersection may form a streaming layer along the interior surface of the output flow path, thereby forming a track or virtual capillary (see, e.g., 322 of FIG. 3.). Such immiscible fluid can be any fluid immiscible with the continuous phase of the fluid in the input flow path. The fluid may be aqueous or non-aqueous, air or liquid, etc. For example, the input flow of fluid may comprise aqueous droplets flowing in a continuous phase comprising oil (or other non-aqueous fluid). An oil-immiscible fluid (e.g., aqueous, water, air) may then be introduced such that the aqueous droplets may then travel along the oil-immiscible fluid virtual capillary layer or track as the droplets flow through the output flow path.

The virtual capillary may alter the aperture of the output flow path. The alteration may be achieved by coating the inner wall of the output flow path with the oil-immiscible fluid, or other fluid. The thickness and/or the size of cross-section of this track or virtual capillary may be adjusted in order to accomplish focusing of the droplets, or positioning of the droplets along a particular dimension(s). In some case, the thickness and/or the size of cross-section of this track or virtual capillary is adjusted by adjusting the viscosity and/or surface tension of the oil-immiscible fluid.

In some cases, this disclosure provides droplet-size independent methods of separating and detecting droplets. For example, the virtual capillary may enable detection of droplets, irrespective of the size of the droplets. In a further embodiment, droplets contained in the virtual capillary are similar in size to the droplets in the input flow channel. In some cases, the virtual capillary may enable detection of a population of droplets of different sizes (such as polydispersed droplets) and/or of different shapes. In some cases, the droplets flow along the virtual capillary to a detection region and are detected. In some other cases, the droplets flow along the virtual capillary in a single file and substantially centered, independent of their sizes.

A droplet can be detected by detecting or sensing the presence or absence of a droplet. In some examples, a droplet is detected by detecting or sensing the droplet or a sample or sample partition in the droplet, such as, for example, with the aid of a signal emanating or detected from the droplet. In other examples, a droplet is detected by detecting or sending the absence of the droplet or a sample or sample partition in the droplet, such as, for example, by determining whether a signal is absent from the droplet.

In another aspect, the droplets may be formed as multiple emulsions (e.g., double emulsions, triple emulsions, quadruple emulsions, etc.). In some cases, double emulsified droplets are formed with diameters about the diameter of the output flow channel are formed; in other cases, the double-emulsified droplets have diameters that are much shorter than the diameter of the output flow channel.

The droplets described in this disclosure can be useful in many applications. In some cases, they contain target nucleic acid(s) (or partitions thereof) and/or materials necessary to carry out an amplification reaction of the target nucleic acid (e.g., polymerase chain reaction (PCR)). In some cases, the droplets may be heated, or subjected to thermal cycling. This can occur prior to, during, or after droplet separation (e.g. prior to entering the input flow path and/or prior to reaching an intersection region). In many cases, PCR is performed in the droplets; in other cases, a reaction other than a PCR reaction occurs within the droplets.

As used herein the term “about” a certain value encompasses exact value as well as values within ±10% of such value, and includes values within the range of 0 to ±10%, including ±1, 2, 3, 4, 5, 6, 7, 8, 9, and 10% as well as values less than ±1%, such as ±0.1, 0.2, 0.3, 0.4, 0.5, 0.6, 0.7, 0.8, or 0.9%.

### Workflows

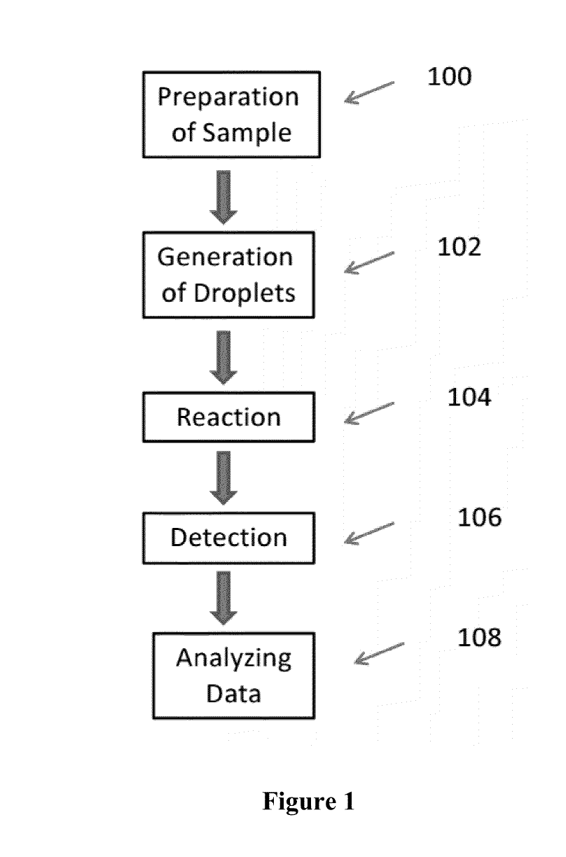

FIG. 1 depicts a workflow for droplet digital PCR (ddPCR) technology. In brief, the workflow may include a sample preparation step 100, followed by a droplet generation step 102, a reaction step 104 (e.g., amplification, PCR, etc.), a detection step 106, and a data analysis step 108. The sample preparation step 100 may involve collecting a sample, such as a clinical or environmental sample, and treating the sample to release associated nucleic acids for PCR amplification. The droplet generation step 102 may involve partitioning the nucleic acids into multiple droplets. In addition to the target nucleic acid for amplification and detection, other reagents, such as a DNA polymerase (e.g., a heat-stable DNA polymerase, such as Taq polymerase), a heat-stable ligase, a dNTPs, magnesium (e.g., Mg²⁺), a primer for a nucleic acid target, among others, may be included. Droplet generation can also involve encapsulating dyes, such as fluorescent molecules, in droplets, for example, with a known concentration of dyes, where the droplets are suspended in an immiscible carrier fluid, such as oil, to form an emulsion. The reaction step 104 may involve subjecting the droplets to a suitable reaction, such as thermal cycling to induce PCR amplification, so that target nucleic acids, if any, within the droplets are amplified to produce additional copies. PCR may be performed by thermal cycling between two or more temperature set points, such as a higher melting (denaturation) temperature and a lower annealing/extension temperature, or among three or more temperature set points, such as a higher melting temperature, a lower annealing temperature, and an intermediate extension temperature, among others. A detection step 106 may involve detecting some signal(s) from the droplets, as an indication as to whether or not there was amplification. Finally, a data analysis step 108 may involve estimating the quantity of target nucleic acid in a sample based on the percentage of droplets in which amplification occurred.

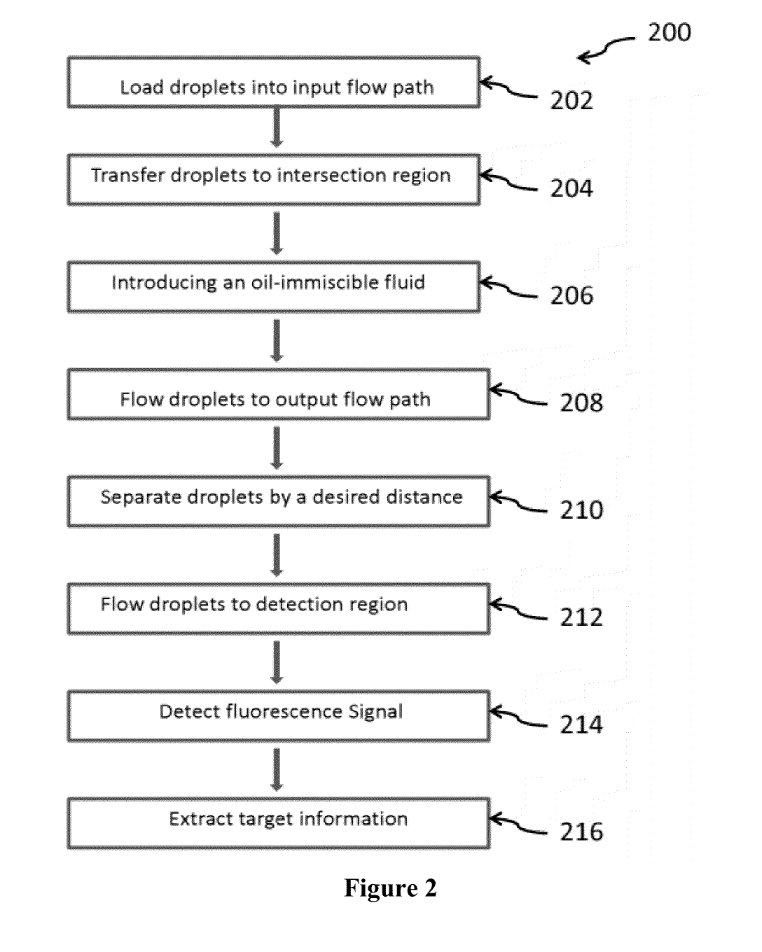

FIG. 2 is a flowchart generally depicting steps of a method of detecting or reading droplets. Although various steps of method 200 are described below and depicted in FIG. 2, the steps need not necessarily all be performed, and in some cases may be performed in a different order than the order shown in FIG. 2. Droplets containing a sample (e.g., nucleic acids) may be loaded into an input flow path 202. The droplets may have been heated or subjected to thermal cycling before entering the input flow path or the intersection. In some cases, the droplets comprise reaction products from a polymerase chain reaction (PCR).

In some embodiments, before entering the input flow path or the intersection, the droplets are heated. In some examples, the droplets are heated by subjecting the droplets to thermal cycling, such as, for example, in a thermal cycler. In other examples, the droplets are heated using a source of conductive, convective and/or radiative heat transfer, such as, for example, one or more resistive heating elements in thermal communication with an input flow path or input region, an infrared (IR) light source, an ultraviolet light source, or fluid with thermal energy that is sufficient to heat the droplets.

The sample-containing droplets may flow or be transferred to an intersection region (204), where they may be contacted with an oil-immiscible fluid (e.g., aqueous fluid or air). In some cases, the droplets and oil-immiscible fluid are introduced to the intersection region simultaneously; in some cases, the droplets and the oil-immiscible fluid are introduced to the intersection region sequentially. After the droplets come in contact with the oil-immiscible fluid, they may form a double emulsion, wherein the droplets comprise an aqueous core enveloped or encapsulated by a non-aqueous fluid that is, in turn, surrounded by the oil-immiscible fluid, which is generally in a continuous phase. In some cases, the oil-immiscible fluid may increase the distance between the droplets (208).

The flow rate of the droplets and the oil-immiscible fluid can be separately controlled. In some cases, the flow of the droplets is controlled by pressure (e.g., vacuum pressure, pump pressure, etc.).

The greater separation may be due to an increase in fluid speed as fluid approaches and travels inside the output flow path. Further downstream of the outlet flow path is at least one detection region. After droplets flow to the detection region (210), the step of detecting a signal (212), such as a fluorescence signal or other signal such as a signal emitted by a radio-isotope, may be carried out. The droplets may be subjected to a stimulus in order to activate the signal, such as fluorescent light or other radiations. For example, the stimulus may be chosen to stimulate emission of fluorescence from one or more fluorescent probes within the droplets. In batch detection applications, the detector and/or the intersection region may be configured to move in a manner that allows an optical scan of the detection region by a detector having a smaller field of view than the entire intersection region.

Detected fluorescence may be analyzed to determine whether or not a particular target nucleotide sequence is present in the droplets 214. Additional information, including but not limited to an estimate of the number or fraction of droplets containing a target molecule, the average concentration of target molecules in the droplets, an error margin, and/or a statistical confidence level, also may be extracted from the collected data. Devices

FIG. 3 is a schematic view of an example droplet spacing and/or focusing device that may, optionally, be used in conjunction with a droplet detector/reader. The device of FIG. 3 can include or be in sensing proximity to a droplet reader (or droplet detector). The device may include an input flow path 300, an intersection region 306, an output flow path 314, a radiation source 318, a detector 320, and a delivery flow path 324. Emulsified droplets 302 in a non-aqueous continuous fluid 303 may enter the detection system through the input flow path 300. The emulsified droplets may be aqueous droplets dispersed within a non-aqueous (e.g., oil) continuous phase 303. In some cases, an aqueous droplet containing a sample (represented by *) is encapsulated by a layer of non-aqueous fluid. In some cases, the droplets within the input flow path are multiple emulsions. For example, the droplets may be present in a double emulsion and may have an aqueous core enveloped or encapsulated by a non-aqueous layer and flow in a continuous non-aqueous fluid. In other cases, the droplets are in a triple emulsion, and may have an aqueous core enveloped or encapsulated by a non-aqueous layer that is further enveloped or encapsulated by an aqueous layer, and the droplets may flow in an aqueous continuous phase. Similarly, the droplets may be a quadruple, quintuple, sextuple, septuple, octuple, or higher-order emulsion. The sample or reaction products may be present in the core of the droplet; however, in some cases the sample or reaction products are present within a particular layer of the emulsion.

Conversely, the droplets may be oil-in-water emulsions. For example, the droplets may have a non-aqueous core and flow in an aqueous continuous phase 303. In this case, an oil is used a focusing/dilution fluid 308. The oil may also form a virtual capillary. The oil-in-water emulsions may also be multiple emulsions. In some cases, the droplets may be a double emulsion and have a non-aqueous core that is enveloped or encapsulated by an aqueous layer (or oil-immiscible) layer, and the droplets flow in a non-aqueous continuous phase.

In FIG. 3 and throughout the present disclosure, the droplets may have different sizes with respect to the size of the output flow path. In some cases, the ratio of the droplet diameter to the output flow path diameter is less than about 3/1, less than about 2.8/1, less than about 2.5/1, less than about 2.2/2, less than about 2.0/1, less than about 1.8/1, less than about 1.5/1, less than about 1.2/1, less than about 1/1, less than about 0.8/1, less than about 0.5/1, less than about 0.3/1, less than about 0.2/1, or less than about 0.1/1. In some cases, the ratio is at least about 3/1, at least about 2.8/1, at least about 2.5/1, at least about 2.2/2, at least about 2.0/1, at least about 1.8/1, at least about 1.5/1, at least about 1.2/1, at least about 1/1, at least about 0.8/1, at least about 0.5/1, at least about 0.3/1, at least about 0.2/1, at least about 0.1/1. In some cases, the ratio is in a range between about 0.1/1 to about 3/or about 0.5/1 to about 2/1. In a further embodiment, the ratio of the droplet diameter to the output flow path diameter is less than about 1/1, less than about 0.8/1, less than about 0.5/1, less than about 0.3/1, or less than about 0.2/1.

Downstream of the flow path is at least one intersection region 306. The intersection region 306 may be an intersection of one or more input flow paths 300 and one or more delivery flow paths 324. In some cases, there are two, three, four, five, six, or even more intersection regions. The intersection region may be cross-shaped, as indicated in FIG. 3. In other cases, the intersection is T-shaped, Y-shaped, or other configurations.

As shown in FIG. 3, the two paths are substantially perpendicular. However, a variety of angles can be constructed. The angel may be at least 1 degree, at least 2 degree, at least 5 degree, at least 10 degree, at least 15 degree, at least 20 degree, at least 25 degree, at least 30 degree, at least 35 degree, at least 40 degree, at least 45 degree, at least 50 degree, at least 55 degree, at least 60 degree, at least 65 degree, at least 70 degree, at least 75 degree, at least 80 degree, at least 85 degree, at least 90 degree, at least 95 degree, at least 100 degree, at least 105 degree, at least 110 degree, at least 115 degree, at least 120 degree, at least 125 degree, at least 130 degree, at least 135 degree, at least 140 degree, at least 145 degree, at least 150 degree, at least 155 degree, at least 160 degree, at least 165 degree, at least 170 degree, or at least 175 degree. In some cases, the angel may be about 1, 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, or 175 degree. In addition, there may be one, two, three, four, five, six, or even more delivery flow paths, each of which may independently have an angle with respect to the input flow path. Each delivery flow path may independently contain an oil or an oil-immiscible fluid. In some cases, an oil or an oil-immiscible fluid is delivered alternatively along a droplet flow path. In some case, an oil and an oil-immiscible fluid are delivered simultaneously to an intersection region along a droplet flow path through two separate delivery flow paths. In some cases, an oil is delivered consecutively along a droplet flow path through multiple delivery flow paths followed by delivering an oil-immiscible fluid through at least one separate delivery flow path. In some cases, an oil-immiscible fluid is delivered consecutively to a droplet flow path through multiple delivery flow paths followed by delivering an oil through at least one separate delivery flow path.

Upon reaching the intersection region 306, the droplets may encounter an oil-immiscible fluid 308 (e.g., an aqueous fluid, air). When the oil-immiscible fluid 308 is an aqueous fluid, the aqueous fluid may envelop or encapsulate the emulsified droplets 302 to form droplets 310 within a double, triple or other multiple emulsions. The droplets may comprise an aqueous core, that is enveloped or encapsulated by a non-aqueous layer; and the droplets may travel through a non-aqueous continuous phase 312. The continuous phases 303 and 312 may have the same or substantially similar composition. The encapsulation may increase the stability of the droplets compared to the droplets in the input flow path 300. The stability of droplets after entering the output flowpath may increase by at least 20%, at least 30%, at least 40%, at least 50%, at least 60%, at least 70%, at least 80%, or at least 90%, compared with the stability of the droplets in the input path. In addition, the envelopment or encapsulation may prevent release of components from the aqueous phase of the droplets, which may help preserve the integrity of information from a prior step (such as a prior PCR amplification).

The flow rate of droplets 302 and the oil-immiscible fluid 308 may be independently controlled. In some cases, the ratio of droplets 302 flow rate/oil-immiscible fluid 308 flow rate is at least 1/10, 1/9, 1/8, 1/7, 1/6, 1/5, 1/4, 1/3, 1/2, 1/1, 2/1, 3/1, 4/1, 5/1, 6/1, 8/1, 10/1 or even higher. In some cases, the ratio of droplets 302 flow rate/oil-immiscible fluid 308 flow rate is no more than 1/10, 1/9, 1/8, 1/7, 1/6, 1/5, 1/4, 1/3, 1/2, 1/1, 2/1, 3/1, 4/1, 5/1, 6/1, 8/1, or 10/1. In some cases, the ratio of droplets 302 flow rate/oil-immiscible fluid 308 flow rate is about 1/10, 1/9, 1/8, 1/7, 1/6, 1/5, 1/4, 1/3, 1/2, 1/1, 2/1, 3/1, 4/1, 5/1, 6/1, 8/1, 10/1, or 50/1. In some cases, the ratio of droplets 302 flow rate/oil-immiscible fluid 308 flow rate is in a range of 10/1 to 1/10

The fluidic properties of oil-immiscible fluid 308, such as an aqueous fluid, can be modified to improve separation and/or centering of droplets in the output flow path and detection region. For example, without being limiting, additives can be added to increase viscosity, surface tension and/or to coat surfaces of the fluid to prevent undesired droplet loss or contamination (e.g. broken droplets or coalescence) and/or improve separation. The additives may include a surfactant, such as a copolymer of polypropylene oxide and polyethylene oxide, and/or a viscosity-enhancing agent, such as glycerol. In the case of an aqueous oil-immiscible fluid 308, components including glycerol, a surfactant, an antimicrobial agent and an antifoaming agent etc., can be added to eliminate a potential need to add such agents to the waste reservoirs during instrument or experiment set up.

The use of aqueous fluid as the oil-immiscible fluid 308 may create a virtual capillary, represented by 322, that may likewise comprise aqueous fluid. In some cases, the fluid in the delivery path 308 is oil that creates a virtual capillary comprising oil 322. In general, the virtual capillary 322 may have an outside layer 316 which covers the inside wall of the output flow path 314 and is composed substantially of aqueous continuous fluid 308 introduced at the intersection region. In a further embodiment, these droplets are substantially centered. The centering can occur regardless or irrespective of the sizes of the droplets or the variability in size of the droplets.

The formation of the virtual capillary 322 may effectively reduce the inner diameter of the output flow path 314, which can lead to an increased flow rate of droplets 310 and fluid in the output flow path 314. The virtual capillary may enable better separation between droplets 310. The average distance of droplets 310 in the output flow path may be greater than the average distance of droplets 302 in the input flow path. In some cases, the average distance of droplets 310 in the output flow path may be at least 1.1, 1.2, 1.5, 2, 5, 10, 15, 20, 25, 30, 50, 100, 1000, 10,000, 100,000, 1 million, or even more times the average distance of droplets 302 in the input flow path. In some cases, the average distance of droplets 310 in the output flow path may be about 1.1, 1.2, 1.5, 2, 5, 10, 15, 20, 25, 30, 50, 100, 1000, 10,000, 100,000, or 1 million times the average distance of droplets 302 in the input flow path.

Furthermore, the virtual capillary 322 may accommodate droplets of varying sizes, therefore, avoiding the need to change output flow path 314 based on the size of incoming droplets 310. The virtual capillary 322 may also help center or focus the droplets 310. In some cases, the virtual capillary reduces contact between the droplets 310 and the inner surface of the output flow path 314. In some cases, the virtual capillary prevents the droplets 310 from contacting the inner surface of the output flow path 314 altogether. In some cases, the ratio of the distance of the outside layer of a droplet 310 to one side of an output flow path 314 and that to the opposite side of the output flow path is within the range of about 0.2 to 5. The ratio may be no more than 5, 4.5, 4, 3.5, 3, 2.5, 2, 1.5, or 1. Alternatively, the ratio may be at least 0.2, 0.3, 0.4, 0.5, 0.6, 0.7, 0.8, or 0.9. The ratio may be about 5, 4.5, 4, 3.5, 3, 2.5, 2, 1.5, or 1.

The formation of the virtual capillary may be controlled by the relative viscosity of the oil-immiscible fluid 308 (Vo) with respect to the non-aqueous fluid 303 (Va). A greater difference in viscosity between the two fluids (e.g., 303, 308) may better enable the formation of the virtual capillary. In some cases, the relative viscosity (Vo/Va) is more than about 1, more than about 1.2, more than about 1.5, more than about 1.8, more than about 2.0, more than about 2.5, more than about 3.0, more than about 3.5, more than about 4.0, more than about 4.5, more than about 5.0, or even higher. In some cases, the relative viscosity (Vo/Va) is less than about 10, less than about 5, less than about 3, less than about 2.5, less than about 2, less than about 1.5. In some cases, the relative viscosity (Vo/Va) is in a range of 1 to 3, 1 to 5, or 1 to 10. In some embodiments, this disclosure provides devices that contain a detection region for detecting, analyzing, or otherwise evaluating the droplets. The detection region may be part of the same device as the droplet spacing region and/or the droplet centering/focusing region. However, in some cases, the detection region is present in a separate device. In some cases, the separate device is connected to the output flow path by a connector (e.g., tube, capillary, channel, etc.).

The detection region or a portion thereof can be operably coupled to or in sensing communication with a detector for detecting the presence or absence of the droplets, or a sample or sample partition in the droplets. A detector can be in sensing communication with the detection region or a portion thereof through one or more intermediate elements, such as optical elements (e.g., lenses, mirrors). The detector can be an optical detector, electrostatic detector, electrochemical detector, or a combination thereof. In some examples, the detector is an optical detector. As an alternative, the detector is an electrostatic detector, such as a field effect transistor (FET) based detector that senses charge, for example.

When the droplets reach the detection region, the droplets may be contacted with an excitation radiation (e.g., light) from a radiation source 318, which may include at least one wavelength chosen to excite the fluorescent probe(s) known to be present in the reagents within the droplets. The radiation source 318 may be a laser, an LED, or any other suitable radiation sources. The radiation may be transferred to the detection region through free space or through one or more optical fibers. Furthermore, the radiation may be focused, diverged, split, filtered, and/or otherwise processed before reaching the detection region.

The fluorescence scattered from the droplets in the detection region may be detected by a detector 320. The fluorescence may be transferred to the detector 320 with or without passing through one or more intermediate optical elements such as lenses, apertures, filters, or the like. The fluorescence also may or may not be transferred to the detector 320 through one or more optical fibers.

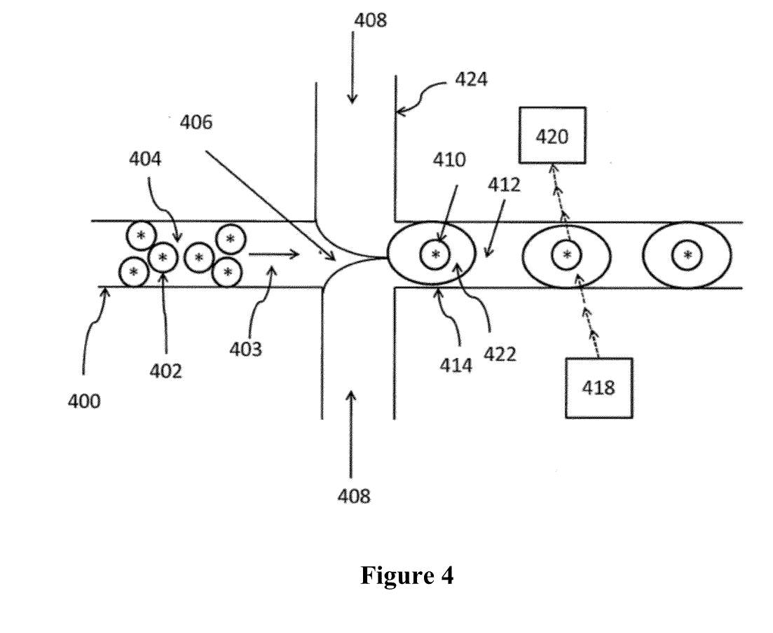

FIG. 4 is a schematic view of an exemplary droplet spacing and/or focusing device, in accordance with an embodiment of the invention. The device may include an input flow path 400, an intersection region 406, an output flow path 414, a radiation source 418, a detector 420, and a delivery flow path 424. Emulsified droplets 402 in a non-aqueous fluid may enter the detection system through the input flow path 400. The emulsified droplets may be aqueous droplets dispersed within a non-aqueous (e.g., oil) continuous phase 403. Conversely, the droplets may be oil-in-water emulsions as described herein.

After encountering the oil-immiscible fluid 408, double emulsified droplets 410 may be formed near or in the output flow path. Diameters of these droplets may be substantially similar to the diameter of the output flow path 414. In some case, the diameters of the double-emulsified droplets are at least about 60%, about 70%, about 80%, about 90%, about 95%, about 98%, or even a higher, compared to the length of the diameters of the output flow path 414.

An appropriate choice of the viscosity of oil-immiscible fluid 408 may allow control of the size and/or formation of double emulsified droplets 410. In some cases, the ratio of the viscosity of oil-immiscible fluid 408 to the viscosity of non-aqueous continuous fluid 403 may be less than about 2, less than about 1.5, less than about 1.2, less than about 1, less than about 0.8, less than about 0.6, less than about 0.5, less than about 0.2, less than about 0.1, less than about 0.05. In some cases, ratio of the viscosity of oil-immiscible fluid 408 to the viscosity of non-aqueous continuous fluid 403 may be in a range of about 1.5 to about 0.01, about 1.2 to about 0.1, about 1 to about 0.1 or about 0.5 to about 0.1. In some cases, a lower viscosity ratio may better aid the development of droplets within a double-emulsion 410.

After passing the intersection region 406, the double emulsified droplets may exit through output flow path 414 in a single file. The emulsified droplets 414 may comprise a non-aqueous layer 422. In some case, the non-aqueous layer 422 may prevent or reduce the likelihood of droplets break up and/or coalesce. In some cases, 422 may have the same or substantially similar composition as 403. Droplets 410 may travel through a continuous phase 412, irradiated by 418 and detected by 420. In some case, the continuous phase 412 is substantially composed of the oil-immiscible fluid 408.

Systems and steps for performing droplet digital PCR (ddPCR) have been described in a number of patent applications, including U.S. Patent Publication Nos. 2011/0092373 to Colston et al., 2011/0092376 to Colston et al., 2011/0217712 to Hiddessen et al., 2011/0311978 to Makarewicz et al., and 2011/0092392 to Colston et al., each of which is entirely incorporated herein by reference.

In some situations, droplets, once formed, are stored in a plate or chip containing one or more wells. The plate can contain, for example, 6, 24, 96, 384, 1536 or more wells. Each well can contain a single droplet or multiple droplets. In some examples, the plate, including the droplets in the wells, is subjected to thermal cycling to facilitate nucleic acid amplification (e.g., PCR).

The one or more droplets in the wells can be individually retrieved and directed to a device of the present disclosure, such as the device of FIG. 3. In some examples, a well is accessed using a tip (e.g., syringe tip, pipette tip) that is in fluid communication with a fluid flow path of the device, and directed to the fluid flow path using negative pressure (or suction) applied to the tip, such as, for example, with the aid of a pumping system. The one or more droplets can then be detected with a droplet reader (or droplet detector). A subsequent well of the plate can then be accessed by the tip to retrieve one or more additional droplets, which can be directed to the fluid flow path using negative pressure and detected using the droplet detector.

The tip can be washed upon accessing all of the wells or between accessing individual wells of the plate. For example, the tip can access a well to retrieve one or more droplets, washed, subsequently used to access another well to retrieve one or more additional droplets, subsequently washed, and so on. The tip can be washed using a wash solution, which can include an oxidizing agent. The wash solution can be in a bath or chamber, and the tip can be washed by dipping the tip in the bath or chamber. In some examples, the wash solution includes sodium hypochlorite, calcium hypochlorite, peroxides (e.g., hydrogen peroxide), sodium percarbonate, sodium perborate, sodium dithionite, sodium borohydride, or combinations thereof. In an example, the wash solution includes bleach. In some cases, the tip is washed by running a fluid down the tip coaxially (e.g., top-down across the outside of the tip). The fluid can be a sheath fluid (e.g., oil) or wash solution, for example.

### Oil-Immiscible Fluids

Oil-immiscible fluids (e.g., aqueous, air), provided by this disclosure, can serve multiple purposes. In some cases, an oil-immiscible fluid comprises a sample and makes up the core of a droplet. In some cases, an oil-immiscible fluid is used as a dilution fluid to dilute the number of droplets in a channel or tube. In other cases, an oil-immiscible fluid is a spacer fluid that is used to modulate the spacing between droplets. In some cases, an oil-immiscible fluid is used to focus a stream of droplets, or to center them within a channel or capillary, or other tube. In some cases, an oil-immiscible fluid is used to prevent droplets from contacting the surface of a channel or capillary, or other tube. In some cases, a virtual capillary, as described herein, may comprise an oil-immiscible fluid.

An oil-immiscible fluid may be delivered to an intersection region through at least one delivery channel. Generally, the oil-immiscible fluid is immiscible with the continuous phase of the emulsion in an input flow path. In some cases, the emulsion within an input flow path is an emulsion of dispersed aqueous droplets flowing within a continuous non-aqueous phase; other emulsions are described herein and are known in the art. When the oil-immiscible fluid contacts a population of emulsified droplets at (or near) an intersection region, the droplets may become enveloped or encapsulated by the oil-immiscible fluid and the encapsulated droplets may flow in the oil-immiscible fluid in an output flow path.

In some embodiments, the oil-immiscible fluid is an aqueous fluid (e.g., water). The use of an aqueous fluid as a spacer/focusing fluid may reduce the cost of operating a droplet detector. Furthermore, the use of an aqueous fluid as a spacer/focusing fluid may reduce the amount of oil waste. In addition, the aqueous fluid may contain additives to adjust chemical and/or physical properties, such as viscosity, surface tension, density, antibacterial property, among others. In some embodiments, the aqueous fluid contains at least one surfactant and/or at least one viscosity-enhancing agent. In some cases, the aqueous fluid contains at least 1, 2, 3, 4, 5, 6, 7, 8, 9, or 10 different surfactants and/or at least 1, 2, 3, 4, 5, 6, 7, 8, 9, or 10 different viscosity-enhancing agents.

In exemplary embodiments, an aqueous fluid-in-oil emulsion with an oil as continuous phase is delivered through an input flow path. The emulsion may contain emulsified droplets. The core of the droplets may be aqueous fluid and may contain at least one surfactant and/or at least one viscosity-enhancing agent. The droplets then may contact an oil-immiscible fluid at or near an intersection region. The surfactant and/or viscosity-enhancing agent in the oil-immiscible fluid may be the same as, or different from, the surfactant and/or viscosity-enhancing agent in the aqueous phase of emulsified droplets. Furthermore, the amount of surfactant and/or viscosity-enhancing agent may be the same as, or different from, the amount in the emulsified droplets. In some cases, both the aqueous core of the droplets and the oil continuous phase contain a surfactant. In some other cases, the aqueous core of the droplets contains a surfactant and the oil continuous phase does not. In some cases, the oil-immiscible fluid contains a surfactant. In some cases, the oil-immiscible fluid does not contain a surfactant.

### Non-Aqueous Fluids

A non-aqueous fluid can serve as a carrier fluid forming a continuous phase in the input flow path. The non-aqueous fluid may be referred to as an oil phase comprising at least one oil, but may include any liquid (or liquefiable) compound or mixture of liquid compounds that is immiscible with water. The oil may be synthetic or naturally occurring. The oil may or may not include carbon and/or silicon, and may or may not include hydrogen and/or fluorine. The oil may be lipophilic or lipophobic. In other words, the oil may be generally miscible or immiscible with organic solvents. Exemplary oils may include at least one silicon oil, mineral oil, hydrocarbon oil, fluorocarbon oil, vegetable oil, soybean oil, or a combination thereof, among others.

In some cases, the oil is a fluorinated oil, such as a fluorocarbon oil, which may be a perfluorinated organic solvent. A fluorinated oil can be a base (primary) oil or an additive to a base oil, among others. Exemplary fluorinated oils that may be suitable are sold under the trade name Fluorinert™ (3M), including, in particular, Fluorinert™ Electronic Liquid FC-3283, FC-40, FC-43, and FC-70. Another example of an appropriate fluorinated oil is sold under the trade name Novec™ (3M), including Novec™ HFE 7500 Engineered Fluid, which is 3-ethoxy-1,1,1,2,3,4,4,5,5,6,6,6-dodecafluoro-2-trifluoromethyl-hexane. In some cases, the fluorine-containing compound is CF₃CF₂CF₂OCH₃, sold as Novec™ HFE 7000. In some cases, the fluorine-containing compound is 2,2,3,3,4,4,4-heptafluoro-1-butanol, CF₃CF₂CF₂CH₂OH. In some cases, the fluorinated oil is perfluorocarbon, such as perfuorooctane or perfluorohexane. In some cases, the fluorine-containing compound is a partially fluorinated hydrocarbon, such as 1,1,1-trifluorooctane or 1,1,1,2,2-petantafluorodecane.

The silicon oil may comprise polydimethylsiloxane. In further embodiments, the polydimethylsiloxane has the viscosity of at least about 40,000 centistokes (cS), or at least about 41,000 cS, or at least about 42,000 cS, or at least about 43,000 cS, or at least about 44,000 cS, or at least about 45,000 cS, or at least about 46,000 cS, or at least about 47,000 cS, or at least about 48,000 cS, or at least about 49,000 cS, or at least about 50,000 cS, or at least about 51,000 cS, or at least about 52,000 cS, or at least about 53,000 cS, or at least about 54,000 cS, or at least about 55,000 cS, or at least about 56,000 cS, or at least about 57,000 cS, or at least about 58,000 cS, or at least about 59,000 cS, or at least about 60,000 cS.

In some cases, the polydimethylsiloxane has a mean molecular weight (Mw) of at least about 800 g/mol, or at least about 850 g/mol, or at least about 900 g/mol, or at least about 1000 g/mol, or at least about 1050 g/mol, or at least about 1100 g/mol, or at least about 1200 g/mol, or at least about 1250 g/mol, or at least about 1300 g/mol, or at least about 1350 g/mol, or at least about 1400 g/mol, or at least about 1450 g/mol, or at least about 1500 g/mol.

In some cases, the silicon oil comprises cyclomethicone. In further embodiments, the cyclomethicone has the viscosity of at least about 5,000 cS, or at least about 5200 cS, or at least about 5400 cSs, or at least about 5600 cS, or at least about 5800 cS, or at least about 6000 cS, or at least about 6200 cS, or at least about 6400 cS, or at least about 6600 cS, or at least about 6800 cS, or at least about 7000 cS.

In some cases, the silicon oil comprises polydiethylsiloxane, poly(di-n-propyl) siloxane, and/or poly(di-i-propyl)siloxane. In some cases, the silicone oil is silanol-terminated. In some cases, the percentage of silanol groups per silicon atom is at least about 0.1%, or at least about 0.2%, or at least about 0.3%, or at least about 0.4%, or at least about 0.5%, or at least about 0.6%, or at least about 0.7%, or at least about 0.8%, or at least about 0.9%, or at least about 1.0%.

### Surfactants

Generally, a surfactant is a surface-active substance capable of reducing the surface tension of a liquid in which it is present. A surfactant, which also or alternatively can be described as a detergent and/or a wetting agent, can incorporate both a hydrophilic portion and a hydrophobic portion, which can collectively confer a dual hydrophilic-hydrophobic character on the surfactant. A surfactant can, in some cases, be characterized according to its hydrophilicity relative to its hydrophobicity.

The present disclosure provides surfactants that can be ionic or a non-ionic. In some cases, the ionic or nonionic surfactants are block copolymers. The block copolymer may be comprised of at least 2, 3, 4, 5, 6, 7, 8, 9, 10, 12, 15, or even 20 separate blocks. Each block may have different molecular weight, topology, hydrophilicity, hydrophobicity, and chain length independent of other blocks. In some embodiments, the surfactant is a block copolymer of polypropylene oxide and polyethylene oxide. More particularly, the surfactant may be a block copolymer of polypropylene oxide and polyethylene oxide sold under the trade names Pluronic® and Tetronic® (BASF). In some embodiments, the surfactant may be a nonionic block copolymer of polypropylene oxide and polyethylene oxide sold under the trade name Pluronic® F-68. In some cases, the surfactant may be a water-soluble and/or hydrophilic fluorosurfactant.

Exemplary fluorinated surfactants include fluorinated polyethers, such as carboxylic acid-terminated perfluoropolyethers, carboxylate salts of perfluoropolyethers, and/or amide or ester derivatives of carboxylic acid-terminated perfluoropolyethers. Exemplary perfluoropolyethers are commercially available under the trade name Krytox® (DuPont), such as Krytox® FSH, the ammonium salt of Krytox® FSH (KRYTOX-AS″), or a morpholino derivative of Krytox® FSH (KRYTOX-M), Zonyl® (DuPont), such as Zonyl® FSN fluorosurfactants, among others. Other fluorinated polyethers that may be suitable include at least one polyethylene glycol (PEG) moiety. Several exemplary examples are shown in Scheme 1.

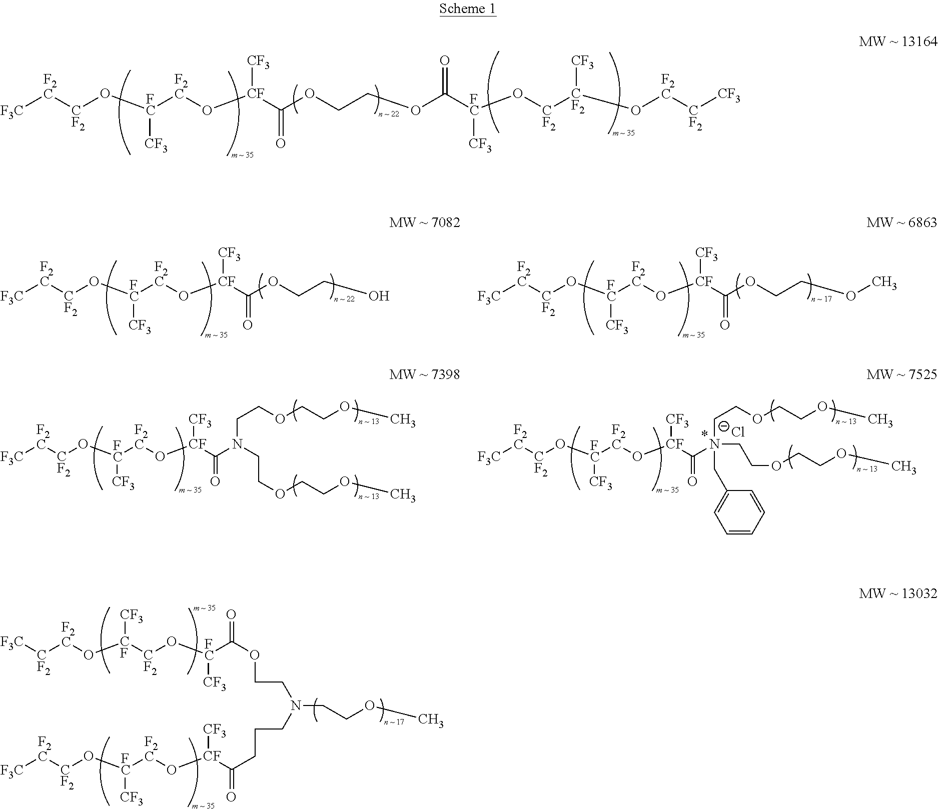

In some cases, the surfactant may include polysorbate 20 (sold under the trade name Tween® 20 by ICI Americas, Inc.).

A fluid (e.g. oil-immiscible or non-aqueous fluid) may include one or more surfactants, each of which may be disposed/dissolved in the fluid prior to, during, and/or after emulsified droplet formation. The surfactants may include a nonionic surfactant, an ionic surfactant (a cationic (positively-charged) or anionic (negatively-charged) surfactant), or any combination thereof. Exemplary anionic surfactants that may be suitable include carboxylates, sulphonates, phosphonates, and so on.

A fluid may comprise a primary surfactant, such as a fluorinated polyether, and at least one additional surfactant, to modify one or more physical properties of the fluidic phase. The ratio of primary surfactant to the at least one additional surfactant may be at least 2:1, at least 3:1, at least 4:1, at least 5:1, at least 6:1, at least 7:1, at least 8:1, at least 9:1, at least 10:1, at least 12:1, at least 15:1, at least 20:1, at least 25:1, at least 30:1, at least 40:1, at least 50:1, or at least 100:1. Alternatively, in some cases, the ratio of primary surfactant to the at least one additions surfactant is no more than these ratios.

Surfactant(s) may be added to the ddPCR workflow (FIG. 1) at any stage. In some cases, surfactant is added before droplet generation, for example, during step 100. In some cases, surfactant is added during droplet generation, for example, during step 102. In some cases, surfactant is added before, during, or after reaction step 104. One or multiple surfactants may be added. When multiple surfactants are added, they may be added the same time or they may be added at different stages of the workflow. When only one surfactant is added, it may be added once or multiple times during the workflow. In some cases, one surfactant is added at droplet generation step 102 and the same surfactant is added at detection step 106. In some cases, one surfactant is added at droplet generation step 102 and a different surfactant is added at detection step 106. There may be various ways of introducing a surfactant in the detection step. For example, a surfactant may be mixed with an—oil immiscible fluid 308 and delivered through a delivery flow path 324 as shown in FIG. 3.

An oil-immiscible fluid or a non-aqueous fluid may comprise at least one surfactant. The amount of surfactant, individually or collectively, may be at least 0.001%, 0.05%, 0.1%, at least 0.2%, at least 0.3%, at least 0.4%, at least 0.5%, at least 0.6%, at least 0.7%, at least 0.8%, at least 0.9%, at least 1.0%, at least 1.5%, at least 2.0%, at least 2.5%, at least 3.0%, at least 3.5%, at least 4.0%, at least 5.0%, at least 6.0%, at least 7.0%, at least 8.0%, at least 9.0%, at least 10%, or at least 15% of the total weight. The amount of surfactant, individually or collectively, may be less than 0.05%, less than 0.1%, less than 0.2%, less than 0.3%, less than 0.4%, less than 0.5%, less than 0.6%, less than 0.7%, less than 0.8%, less than 0.9%, less than 1.0%, less than 1.5%, less than 2.0%, less than 2.5%, less than 3.0%, less than 3.5%, less than 4.0%, less than 5.0%, less than 6.0%, less than 7.0%, less than 8.0%, less than 9.0%, less than 10%, or less than 15% of the total weight. The amount of surfactant, individually or collectively, may be about 0.05%, about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 3.5%, about 4.0%, about 5.0%, about 6.0%, about 7.0%, about 8.0%, about 9.0%, about 10%, or about 15% of the total weight.

### Viscosity-Enhancing Agents

A viscosity-enhancing agent (or thickening agent) is an agent which can increase the viscosity of a fluid upon mixing with the fluid. Besides increasing viscosity, the addition of a viscosity-enhancing agent may also lead to an increase of fluid density.

Any agent capable of enhancing viscosity of an oil-immiscible fluid can be referred as a viscosity-enhancing agent herein. Without being limiting, such an agent includes polysaccharides and polyols. The viscosity-enhancing agent may be naturally derived or synthesized. In some cases, the viscosity-enhancing agent is glycerol.

An oil-immiscible fluid or a non-aqueous fluid may comprise at least one viscosity-enhancing agent. The amount of viscosity-enhancing agent, individually or collectively, may be at least 0.001%, 0.01%, 0.05%, 0.5%, at least 1%, at least 1.5%, at least 2.0%, at least 2.5%, at least 3.0%, at least 3.5%, at least 4.0%, at least 5.0%, at least 6.0%, at least 7.0%, at least 8.0%, at least 9.0%, at least 10.0%, at least 12.0%, at least 15.0%, at least 20%, at least 25%, at least 30%, at least 40%, or at least 50% of the total weight. The amount of viscosity-enhancing agent, individually or collectively, may be less than 0.5%, less than 1%, less than 1.5%, less than 2.0%, less than 2.5%, less than 3.0%, less than 3.5%, less than 4.0%, less than 5.0%, less than 6.0%, less than 7.0%, less than 8.0%, less than 9.0%, less than 10.0%, less than 12.0%, less than 15.0%, less than 20%, less than 30%, less than 40%, or less than 50% of the total weight. The amount of viscosity-enhancing agent, individually or collectively, may be about 0.5%, about 1%, about 1.5%, about 2.0%, about 2.5%, about 3.0%, about 3.5%, about 4.0%, about 5.0%, about 6.0%, about 7.0%, about 8.0%, about 9.0%, about 10.0%, about 12.0%, about 15.0%, about 20%, about 30%, about 40%, or about 50% of the total weight.

One or more viscosity-enhancing agent may be added to the ddPCR workflow (FIG. 1) at any stage. In some cases, a viscosity-enhancing agent is added before droplet generation, for example, during step 100. In some cases, a viscosity-enhancing agent is added during droplet generation, for example, during step 102. In some cases, a viscosity-enhancing agent is added before, during, or after reaction step 104. When multiple viscosity-enhancing agents are added, they may be added the same time or they may be added at different stages of the workflow. When only one viscosity-enhancing agent is added, it may be added once or multiple times during the workflow. In some cases, one viscosity-enhancing agent is added at droplet generation step 102 and the same surfactant is added at detection step 106. In some cases, one surfactant is added at droplet generation step 102 and a different viscosity-enhancing agent is added at detection step 106. There are may be various ways of introducing a viscosity-enhancing agent in the detection step. For example, a viscosity-enhancing agent may be mixed with an—oil immiscible fluid 308 and delivered through a delivery flow path 324 as shown in FIG. 3.

### Antimicrobial Agents

An antimicrobial agent (e.g., antibacterial, antibiotic, antifungal agent) is a compound or substance that kills or slows down the growth of bacteria or fungi. Antimicrobial agents may be classified on the basis of chemical/biosynthetic origin into natural, semisynthetic, and synthetic. Without being limiting, the antimicrobial agents includes beta-lactams, penicillins, aminoglycosides, sulfonamides, quinolones, and oxazolidinones, polyene antifungals, imidazole, triazole, and thiazole antifungals, allylamines, echinocandins, among others. The bacteria includes Gram-positive and Gram-negative bacteria. Exemplary examples of bacteria include, but are not limited to, Actinomyces, Bacillus, Clostridium, Corynebacterium, Enterococcus, Gardnerella, Lactobacillus, Listeria, Mycobacterium, Mycoplasma, Nocardia, Propionibacterium, Staphylococcus, Streptococcus, Streptomyces, Acetobacter, Borrelia, Bortadella, Burkholderia, Campylobacter, Chlamydia, Enterobacter, Escherichia, Fusobacterium, Helicobacter, Hemophilus, Klebsiella, Legionella, Leptospiria, Neisseria, Nitrobacter, Proteus, Pseudomonas, Rickettsia, Salmonella, Serratia, Shigella, and Yersinia. Exemplary examples of fungi include, but are not limited to, Amethyst Deceiver, Agaricus geesterani, Birch Woodwart, Clavulinopsis helveola, Eyelash Cup Fungus, Fringed Earthstar, Giant Polypore, Hypoxylon serpens, Beef-steak Fungus, Butter Cap, Dead Mans Fingers, Dyer's Polypore, Emetic Russula, King Bolete, Meadow Waxcap, Artist's Fungus, Bovine Bolete, Candlesnuff Fungus, Carbon Balls, Club Foot, White Coral Fungus, White Saddle, Witch's Butter, Wolf's Milk, Wood Hedgehog, Wrinkled Shield, Yellow false truffle, and Yellow Stagshorn.

An oil-immiscible fluid or a non-aqueous fluid may comprise at least one antimicrobial agent. In some cases, the amount of antimicrobial agent, individually or collectively, may be at least 0.001%, at least 0.01%, at least 0.05%, at least 0.1%, at least 0.2%, 0.5%, at least 1%, at least 1.5%, at least 2.0%, at least 2.5%, at least 3.0%, at least 3.5%, at least 4.0%, at least 5.0%, at least 6.0%, at least 7.0%, at least 8.0%, at least 9.0%, or at least 10.0% of the total weight. In some cases, the amount of antimicrobial agent, individually or collectively, may be less than 0.5%, less than 1%, less than 1.5%, less than 2.0%, less than 2.5%, less than 3.0%, less than 3.5%, less than 4.0%, less than 5.0%, less than 6.0%, less than 7.0%, less than 8.0%, less than 9.0%, or less than 10.0% of the total weight. In some cases, the amount of antimicrobial agent, individually or collectively, may be about 0.5%, about 1%, about 1.5%, about 2.0%, about 2.5%, about 3.0%, about 3.5%, about 4.0%, about 5.0%, about 6.0%, about 7.0%, about 8.0%, about 9.0%, or about 10.0% of the total weight.

One or more antimicrobial agent may be added to the ddPCR workflow (FIG. 1) at any stage. In some cases, an antimicrobial agent is added before droplet generation, for example, during step 100. In some cases, an antimicrobial agent is added during droplet generation, for example, during step 102. In some cases, an antimicrobial agent is added before, during, or after reaction step 104. When multiple antimicrobial agents are added, they may be added the same time or they may be added at different stage of the workflow. When only one antimicrobial agent is added, it may be added once or multiple times during the workflow. In some cases, one antimicrobial agent is added at droplet generation step 102 and the same antimicrobial agent is added at detection step 106. In some cases, one antimicrobial agent is added at droplet generation step 102 and a different antimicrobial agent is added at detection step 106. There are may be various ways of introducing an antimicrobial agent before or after the detection step. For example, an antimicrobial agent may be mixed with an—oil immiscible fluid 308 and delivered through a delivery flow path 324 as shown in FIG. 3.

### Anti-Foaming Agents

An anti-foaming agent is a chemical additive that reduces and/or hinders the formation of foam in liquids. In some cases, the anti-foaming agent is oil-based. Examples include, but are not limited to, mineral oil, vegetable oil, white oil or any other oil that is insoluble in the foaming medium. An oil-based anti-foaming agent may also contain a wax and/or hydrophobic silica to boost the performance. Typical waxes are ethylene bis stearamide (EBS), paraffinic waxes, ester waxes and fatty alcohol waxes. In some case, the anti-foaming agent is powder-based. The poder-based anti-foaming agent may be made from silica carrier. In some case, the anti-foaming agent is water-based. Water based anti-foaming agents may comprise different types of oils and waxes dispersed in a water base. The oils may be white oils or vegetable oils and the waxes may be long chain fatty alcohol, fatty acid soaps or esters. In some case, the anti-foaming agent comprises polyethylene glycol and/or ethylene glycol and propylene glycol copolymer. In some case, the anti-foaming agent comprises alkyl polyacrylate.

### Combinations of Agents

A surfactant, a viscosity-enhancing agent, an anti-foaming agent and an antimicrobial agent can be added separately or in any combination to a fluid. In some cases, all four agents are added to an oil-immiscible fluid to mix with droplets coming from an input flow path. In some cases, one agent may have dual functions. For example, a surfactant may also be an anti-foaming agent. A fluid can comprise at least one, at least two, at least three, or all four of a surfactant, a viscosity-enhancing agent, an anti-foaming agent and an antimicrobial agent.

The combined use of a surfactant and a viscosity-enhancing agent may lead to greater separation of droplets in output flow path compared to using the same amount of either agent alone. The above mentioned separation may be increased by at least 20%, at least 30%, at least 40%, at least 50%, at least 60%, at least 70%, at least 80%, at least 90% or even more. This, in turn, may lead to a better signal to noise ratio in the detection step. The signal to noise ratio may be increased by at least 20%, at least 30%, at least 40%, at least 50%, at least 60%, at least 70%, at least 80%, or even more. In other words, there may be a synergistic effect of combining at least one surfactant and at least one viscosity-enhancing agent to improve data quality.

The amount and ratio of surfactant and viscosity-enhancing agent may depend on many factors, for example without being limiting, types of non-aqueous and oil-immiscible fluid, types of surfactant and viscosity-enhancing agent, desired flow rate, and method of detection. In some cases, the surfactant is a block copolymer of polypropylene oxide and polyethylene oxide, and the viscosity-enhancing agent is glycerol. In some embodiments, the amount of polypropylene oxide and polyethylene oxide block copolymer is at least 0.5% and the amount of glycerol is at least 2%. In a further embodiment, the amount of polypropylene oxide and polyethylene oxide block copolymer is at least 1% and the amount of glycerol is at least 5%. In a particular embodiment, the amount of polypropylene oxide and polyethylene oxide block copolymer is 2% and the amount of glycerol is 8%. The weight ratio of viscosity-enhancing agent to surfactant may be greater than or equal to 0.5, 0.6, 0.7, 0.8, 0.9, 1.0, 1.1, 1.2, 1.3, 1.4, 1.5, 1.6 1.7, 1.8, 1.9, 2.0, 2.2, 2.4, 2.6, 2.8, 3.0, 3.5, 4.0, 5.0, or even higher. The weight ratio of viscosity-enhancing agent to surfactant may be less than 0.5, 0.6, 0.7, 0.8, 0.9, 1.0, 1.1, 1.2, 1.3, 1.4, 1.5, 1.6 1.7, 1.8, 1.9, 2.0, 2.2, 2.4, 2.6, 2.8, 3.0, 3.5, 4.0, 5.0, or even higher. The weight ratio of viscosity-enhancing agent to surfactant may be about 0.5, 0.6, 0.7, 0.8, 0.9, 1.0, 1.1, 1.2, 1.3, 1.4, 1.5, 1.6 1.7, 1.8, 1.9, 2.0, 2.2, 2.4, 2.6, 2.8, 3.0, 3.5, 4.0, 5.0, or even higher. In some embodiments, the weight ratio of viscosity-enhancing agent to surfactant is greater than 2. In a further embodiment, the weight ratio of viscosity-enhancing agent to surfactant is greater than 3.

In addition to at least one surfactant and at least one viscosity-enhancing agent, at least one antimicrobial agent may be added to an oil-immiscible fluid. The amount of antimicrobial agent is not particularly limited as long as the addition of the antimicrobial agent can prevent and/or slow down bacterial or fungi growth.

### Emulsions

An emulsion can include droplets of a dispersed phase (e.g., an aqueous phase) disposed in an immiscible continuous phase (e.g., a non-aqueous phase such as an oil phase) that serves as a carrier fluid or continuous fluid for the droplets. Both the dispersed and continuous phases generally are at least predominantly liquid. The emulsion may be a water-in-oil (W/O) emulsion, an oil-in-water (O/W) emulsion or a multiple emulsion (e.g., a W/O/W or a W/O/W/O emulsion, among others). The emulsion may be a double, triple, quadruple, quintuple, sextuple, septuple, octuple, or higher-order emulsion.

Any suitable method and device (or apparatus) can be used to form the emulsion and droplets. Generally, energy input is needed to form the emulsion, such as shaking, stirring, sonicating, agitating, or otherwise homogenizing the emulsion. However, these approaches generally produce polydispersed emulsions, in which droplets exhibit a range of sizes, by substantially uncontrolled generation of droplets. Alternatively, monodispersed emulsions (with a highly uniform size of droplets) may be created by controlled, serial droplet generation with at least one droplet generator. The droplet generator may operate by microchannel flow focusing to generate an emulsion of monodispersed droplets. Other approaches to and structures for droplet generation that may be suitable include those disclosed in U.S. Patent Publication No. 2011/0053798 to Hiddessen et al., published on Mar. 3, 2011; and U.S. Patent Publication No. 2010/0173394 to Colston et al., published on Jul. 8, 2010, each of which publications is entirely incorporated herein by reference.

A surfactant present in the aqueous phase may aid in the formation of emulsified droplets within a non-aqueous phase. The surfactant may do so by physically interacting with both the non-aqueous phase and the aqueous phase, stabilizing the interface between the phases, and forming a self-assembled interfacial layer. The surfactant generally increases the kinetic stability of the droplets significantly, substantially reducing coalescence of the droplets, as well as reducing aggregation. The droplets may be relatively stable to shear forces created by fluid flow during fluidic manipulation. For example, the droplets may be stable to flow rates of at least 5, 10, 15, 20, 25, 20, 35, 40, 45, 50, 60, 70, 80, 90, 100 μL/min, or even a high rate using selected combinations of non-aqueous and aqueous phase formulations in a channel. In some cases, the droplets may be stable to flow rates of no more than 5, 10, 15, 20, 25, 20, 35, 40, 45, 50, 60, 70, 80, 90, or 100 μL/min using selected combinations of non-aqueous and aqueous phase formulations in a channel. In some cases, the droplets may be stable to flow rates of at about 5, 10, 15, 20, 25, 20, 35, 40, 45, 50, 60, 70, 80, 90, or 100 μL/min using selected combinations of non-aqueous and aqueous phase formulations in a channel. The size of channel may be at least 10, 20, 30, 40, 50, 60, 70, 80, 90, 100, 110, 120, 130, 140, 150, 160, 170, 180, 190 200, 210, 220, 230, 240, 250, 260, 270, 280, 290, 300 μm, or even higher. The size of channel may be no more than 10, 20, 30, 40, 50, 60, 70, 80, 90, 100, 110, 120, 130, 140, 150, 160, 170, 180, 190 200, 210, 220, 230, 240, 250, 260, 270, 280, 290 or 300 μm. The size of channel may be about 20, 30, 40, 50, 60, 70, 80, 90, 100, 110, 120, 130, 140, 150, 160, 170, 180, 190 200, 210, 220, 230, 240, 250, 260, 270, 280, 290 or 300 μm.

The resulting droplets may have any suitable shape and size. The droplets may be spherical, when shape is not constrained. In some cases, the average diameter of the droplets may be at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 120, 140, 160, 180, 190, 200, 240, 280, 300, 350, 400, 450, 500, 550, 600, 700, 800 or 900 μm. In some cases, the average diameter of the droplets may be less than 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 120, 140, 160, 180, 190, 200, 240, 280, 300, 350, 400, 450, 500, 550, 600, 700, 800 or 900 μm. In some cases, the average diameter of the droplets may be about 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 120, 140, 160, 180, 190, 200, 240, 280, 300, 350, 400, 450, 500, 550, 600, 700, 800 or 900 μm. The average volume of the droplets may be at least 10 pL, 20 pL, 30 pL, 40 pL, 50 pL, 60 pL, 70 pL, 80 pL, 90 pL, 100 pL, 120 pL, 140 pL, 160 pL, 180 pL, 200 pL, 220, pL, 240 pL, 260 pL, 280 pL, 300 pL, 400 pL, 500 pL, 600 pL, 700 pL, 800 pL, 900 pL, 1 nL, 2 nL, 3 nL, 4 nL, 5 nL, 6 nL, 7 nL, 8 nL, 9 nL, 10 nL, 15 nL, 20 nL, 25 nL, 30 nL, 35 nL, 40 nL, 45 nL, 50 nL, 55 nL, 60 nL, 65 nL, 70 nL, 75 nL, 80 nL, 85 nL, 90 nL, 95 nL, 100 nL, 150 nL, 200 nL, 250 nL, 300 nL, 350 nL, 400 nL, 450 nL, or 500 nL. The average volume of the droplets may be less than 10 pL, 20 pL, 30 pL, 40 pL, 50 pL, 60 pL, 70 pL, 80 pL, 90 pL, 100 pL, 120 pL, 140 pL, 160 pL, 180 pL, 200 pL, 220, pL, 240 pL, 260 pL, 280 pL, 300 pL, 400 pL, 500 pL, 600 pL, 700 pL, 800 pL, 900 pL, 1 nL, 2 nL, 3 nL, 4 nL, 5 nL, 6 nL, 7 nL, 8 nL, 9 nL, 10 nL, 15 nL, 20 nL, 25 nL, 30 nL, 35 nL, 40 nL, 45 nL, 50 nL, 55 nL, 60 nL, 65 nL, 70 nL, 75 nL, 80 nL, 85 nL, 90 nL, 95 nL, 100 nL, 150 nL, 200 nL, 250 nL, 300 nL, 350 nL, 400 nL, 450 nL, or 500 nL. The average volume of the droplets may be about 10 pL, 20 pL, 30 pL, 40 pL, 50 pL, 60 pL, 70 pL, 80 pL, 90 pL, 100 pL, 120 pL, 140 pL, 160 pL, 180 pL, 200 pL, 220, pL, 240 pL, 260 pL, 280 pL, 300 pL, 400 pL, 500 pL, 600 pL, 700 pL, 800 pL, 900 pL, 1 nL, 2 nL, 3 nL, 4 nL, 5 nL, 6 nL, 7 nL, 8 nL, 9 nL, 10 nL, 15 nL, 20 nL, 25 nL, 30 nL, 35 nL, 40 nL, 45 nL, 50 nL, 55 nL, 60 nL, 65 nL, 70 nL, 75 nL, 80 nL, 85 nL, 90 nL, 95 nL, 100 nL, 150 nL, 200 nL, 250 nL, 300 nL, 350 nL, 400 nL, 450 nL, or 500 nL.

### Flow Paths

A flow path may be an elongate passage for fluid travel. A flow path may be a channel, tube, capillary or any other hollow structures allowing the flow of a liquid mixture. The inner side of a flow path may be coated with a hydrophilic or hydrophobic polymer to assist the flow of an emulsion. The choice of inner coating may depend on the continuous phase of an emulsion. In some cases, a hydrophilic polymer is used to coat the inner side of a flow path to assist the flow of an emulsion with an oil continuous phase. In some other cases, a hydrophobic polymer is used to coat the inner side of a flow path to assist the flow of an emulsion with an aqueous continuous phase.

A flow path generally includes at least one inlet, where fluid enters the path, and at least one outlet, where fluid exits the path. The functions of the inlet and the outlet may be interchangeable, that is, fluid may flow through a path in only one direction or in opposing directions, generally at different times. A path may include walls that define and enclose the passage between the inlet and the outlet. A path may, for example, be formed by a tube (e.g., a capillary tube), in or on a planar structure (e.g., a chip), or a combination thereof, among others. A path may or may not branch. A path may be linear or nonlinear; it may be straight or curved. Exemplary curved paths include a path extending along a planar flow direction (e.g., a serpentine path, a C-shaped path), a non-planar flow path (e.g., a helical path to provide a helical flow direction), and others.

In some cases, a flow path has an inner cross-section of at least 0.01, 0.05, 0.1, 0.2, 0.3, 0.4, 0.5, 0.6, 0.7, 0.8, 0.9, 1.0, 1.2, 1.5, 1.8, 2.0, 2.5, 3.0, 3.5, 4.0, 4.5, 5.0, 5.5, 6.0, 7.0, 8.0, 9.0, 10.0, 15.0, 20.0, 25.0, 30.0, 35.0, 40.0, 45.0, 50, 55, or 60 millimeter. In some cases, a flow path has an inner cross-section of less than 0.01, 0.05, 0.1, 0.2, 0.3, 0.4, 0.5, 0.6, 0.7, 0.8, 0.9, 1.0, 1.2, 1.5, 1.8, 2.0, 2.5, 3.0, 3.5, 4.0, 4.5, 5.0, 5.5, 6.0, 7.0, 8.0, 9.0, 10.0, 15.0, 20.0, 25.0, 30.0, 35.0, or 40.0 millimeter. In some cases, a flow path has an inner cross-section of about 0.01, 0.05, 0.1, 0.2, 0.3, 0.4, 0.5, 0.6, 0.7, 0.8, 0.9, 1.0, 1.2, 1.5, 1.8, 2.0, 2.5, 3.0, 3.5, 4.0, 4.5, 5.0, 5.5, 6.0, 7.0, 8.0, 9.0, 10.0, 15.0, 20.0, 25.0, 30.0, 35.0, or 40.0 millimeter. A flow path also may include one or more venting mechanisms to allow fluid to enter/exit without the need for an open outlet. Examples of venting mechanisms include but are not limited to hydrophobic vent openings or the use of porous materials to either make up a portion of the channel or to block an outlet if present.

As described above, a flow path may include at least one input flow path, at least one intersection region, at least one delivery flow path, at least one downstream outlet flow path, and at least one further downstream detection region. In general, fluids from the input and the delivery flow path may exit from the downstream outlet flow path. The downstream outlet flow path may be configured to have a smaller inner diameter than the inner diameter of some or all of input or delivery flow path.

Droplets-containing fluid may flow more rapidly through the output flow path than through the other paths. In some cases, the flow rate of droplets in the output flow path is at least 1.1, 1.15, 1.18, 1.20, 1.25, 1.30, 1.35, 1.40, 1.45, 1.50, 1.55, 1.60, 1.65, 1.70, 1.75, 1.80, 1.85, 1.90, 1.95, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 6.0, 6.5, 7.0, 8.0, 9.0, 10.0, 15.0, 20.0, 30.0, 35.0, 40.0, 45.0, 50.0, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10,000, 20,000, 30,000, 50,000 or even more times the flow rate of droplets in an input flow path. Because of the increase in fluid speed as fluid approaches the outlet flow path, droplets accelerate as they enter the outlet flow path. The average distance of droplets in the outlet flow path may be greater than the average distance of droplets in the input flow path.

The present disclosure provides methods of achieving or controlling droplet separation. In some cases, optimal droplet separation can be achieved without relying on an output flow path with a smaller inner diameter than some of the input and/or delivery path.

The addition of an oil-immiscible fluid to droplets in a non-aqueous continuous fluid along a flow path may create a virtual capillary along the inside of the output flow path. The inner wall of the output flow path may be coated by the oil-immiscible fluid, thus reducing the aperture of the output flow path. The thickness of the oil-immiscible fluid coating the inner wall may be at least 0.01%, at least 0.1%, at least 1% or at least 5% of the diameter of the output flow path. In some cases, the thickness of the oil-immiscible fluid coating the inner wall is in a range of 0.01%-90%, 0.1%-90%, 1%-90%, 5%-90%, 10%-90%, 20%-90%, 30%-90%, 1%-95%, 5%-95%, 10%-95%, 20%-95% or 30%-95% of the diameter of the output flow path. In some case, the aperture of the output flow path is reduced by at least 5%, 6%, 7%, 8%, 9%, 10%, 11%, 12%, 13%, 14%, 15%, 18%, 20%, 25%, 30%, 35%, 40%, 45%, 50%, 55%, 60%, 65%, 70%, 75%, 80%, 85%, 90%, 95% or even more. In some case, the aperture of the output flow path is reduced by about 5%, about 6%, about 7%, about 8%, about 9%, about 10%, about 11%, about 12%, about 13%, about 14%, about 15%, about 18%, about 20%, about 25%, about 30%, about 35%, about 40%, about 45%, about 50%, about 55%, about 60%, about 65%, about 70%, about 75%, about 80%, about 85%, about 90% or about 95%. In some cases, the cross-section of the virtual capillary is less than 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 68%, 66%, 64%, 62%, 60%, 58%, 56%, 54%, 52%, 50%, 48%, 46%, 44%, 42%, 40%, 38%, 36%, 34%, 32%, 30%, 28%, 26%, 24%, 22%, 20%, 18%, 16%, 14%, 12%, 10%, 9%, 8%, 7%, 6%, 5%, 4%, 3%, 2%, or 1% of that of a output flow path. The smaller diameter of the virtual capillary may avoid the need to have a smaller output flow path size than the size of the input and/or the delivery flow path.

The present disclosure may allow for the use of a single-sized output flow path that is capable of accommodating droplets of varying sizes and shapes. In some cases, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of droplets are spherical. In some cases, about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of droplets are spherical. In some cases, at least ______% of droplets have diameters greater than 50% of the diameters of the output flow path. In some cases, at least ______% of droplets have diameters less than 50% of the diameters of the output flow path. In some cases, no more than 10% of droplets have diameters greater than 50% of the diameters of the output flow path. In some cases, no more than 20% of droplets have diameters greater than 50% of the diameters of the output flow path.

### Flow Mechanisms

The flow of droplets and fluids may be controlled by positive pressure, or negative pressure, or a combination of both. For example, positive pressure may be applied to the fluid and droplets at the beginning of a flow path. Under the positive pressure, the fluid and droplets flow through the input flow path, to the intersection region, the output channel, and then the detection region. The positive pressure can come from any source capable of providing positive pressure. Without being limiting, the source of positive pressure includes at least one pump, at least one syringe, or a combination of both. Alternatively, negative pressure can be applied at the end of a flow path to drive the flow of fluid and droplets. Without being limiting, the negative pressure may be from vacuum pressure (e.g., produced by a vacuum pump). The vacuum pump may optionally be attached to at least one control valve and/or device to control the level of negative pressure applied to the system. In addition, combination of positive pressure at the beginning of flow path and negative pressure at the end of flow path may be applied to drive the flow of droplets and fluid.

Fluid flow rate (i.e., speed or velocity of flow) can be influenced by the level of positive and negative pressure applied, the viscosity of a fluid, the coating material inside a flow path, among others. In some cases, the flow rate of droplets in the input flow path may be at least 0.01 μL/min, at least 0.1 μL/min, or at least 1 μL/min. In some cases, the flow rate of droplets in the output flow path may be at least 0.01 μL/min, at least 0.1 μL/min, or at least 1

### Systems

The present disclosure provides systems or kits for detecting emulsified droplets. The system may comprise a detector device and/or an oil-immiscible fluid. In some cases, the system comprises any combination of the following: (a) one or more droplet generators; (b) one or more droplet spacing and/or positioning devices; (c) one or more droplet readers; (d) one or more thermal cycling devices; (e) water-in-oil droplets; (f) oil-in-water droplets; (g) doubly-emulsified droplets comprising an aqueous core enveloped by a non-aqueous core flowing through an aqueous continuous phase; (h) one more additives (e.g., surfactant, viscosity-enhancing agent or antimicrobial agent); and (i) virtual capillaries.

### Clinical Applications

Systems of the present disclosure may be used to perform clinical (and/or forensic) tests related to etiology, pathogenesis, diagnosis, surveillance, and/or therapy monitoring of any suitable infection, disorder, physiological condition, and/or genotype, among others, as illustrated below. Pathogen testing may involve pathogen detection, speciation, and/or drug sensitivity applications, among others.

Each clinical (or non-clinical) test listed below may analyze any suitable aspect of a particular nucleic acid target or set of two or more targets (e.g., clinically related targets) using any suitable amplification methodology. For example, the test may be qualitative, to determine whether or not the target (or each target) is present at a detectable, statistically significant level above background in a sample, or the test may be quantitative, to determine a total presence (i.e., a concentration/copy number) of the target (or each target) in the sample. Alternatively, or in addition, the test may determine a sequence characteristic of a target (such as to determine the identity of a single nucleotide polymorphism (SNP) in the target, whether the target is wild-type or a variant, to genotype the target, and/or the like). Any suitable amplification methodology may be used in performing the tests, such as polymerase chain reaction (PCR), in vitro transcription/translation, self-sustained sequence replication, nucleic acid sequence-based amplification (NASBA) or ligase chain reaction, among others.

Amplification can be performed with any suitable reagents. Amplification can be performed, or tested for its occurrence, in an amplification mixture, which is any composition capable of generating multiple copies of a nucleic acid target molecule, if present, in the composition. An amplification mixture can include any combination of at least one primer or primer pair, at least one probe, at least one replication enzyme (e.g., at least one polymerase, such as at least one DNA and/or RNA polymerase), and deoxynucleotide (and/or nucleotide) triphosphates (dNTPs and/or NTPs), among others.

PCR nucleic acid amplification relies on alternating cycles of heating and cooling (i.e., thermal cycling) to achieve successive rounds of replication. PCR can be performed by thermal cycling between two or more temperature set points, such as a higher melting (denaturation) temperature and a lower annealing/extension temperature, or among three or more temperature set points, such as a higher melting temperature, a lower annealing temperature, and an intermediate extension temperature, among others. PCR can be performed with a thermostable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others.

Any suitable PCR methodology or combination of methodologies can be calibrated utilizing the droplet mixtures disclosed herein, such as allele-specific PCR, assembly PCR, asymmetric PCR, digital PCR, endpoint PCR, hot-start PCR, in situ PCR, intersequence-specific PCR, inverse PCR, linear after exponential PCR, ligation-mediated PCR, methylationspecific PCR, miniprimer PCR, multiplex ligation-dependent probe amplification, multiplex PCR, nested PCR, overlap extension PCR, polymerase cycling assembly, qualitative PCR, quantitative PCR, real-time PCR, RT-PCR, single-cell PCR, solid-phase PCR, thermal asymmetric interlaced PCR, touchdown PCR, or universal fast walking PCR, among others. Digital PCR can refer to PCR performed on portions of a sample to determine the presence, absence, concentration, or copy number of a nucleic acid target in the sample, based on how many of the sample portions support amplification of the target. Digital PCR can be performed as endpoint PCR or as real-time PCR for each of the partitions.

PCR theoretically results in an exponential amplification of a nucleic acid sequence from a sample. By measuring the number of amplification cycles required to achieve a threshold level of amplification, one can theoretically calculate the starting concentration of nucleic acid. In practice, however, there are many factors that make the PCR process non-exponential, such as varying amplification efficiencies, low copy numbers of starting nucleic acid, and competition with background contaminant nucleic acid. Digital PCR is generally insensitive to these factors, since it does not rely on the assumption that the PCR process is exponential. In digital PCR, individual nucleic acid molecules are separated from the initial sample into partitions, then amplified to detectable levels. Each partition then provides digital information on the presence or absence of each individual nucleic acid molecule within each partition. When enough partitions are measured using this technique, the digital information can be consolidated to make a statistically relevant measure of starting concentration for the nucleic acid target (analyte) in the sample.

The concept of digital PCR may be extended to other types of analytes, besides nucleic acids. In particular, a signal amplification reaction may be utilized to permit detection of a single copy of a molecule of the analyte in individual droplets, or to permit data analysis of droplet signals for other analytes. Exemplary signal amplification reactions that permit detection of single copies of other types of analytes in droplets include enzyme reactions.

A primer can be a nucleic acid capable of, and/or used for, priming replication of a nucleic acid template. Thus, a primer is a shorter nucleic acid that is complementary to a longer template. During replication, the primer is extended, based on the template sequence, to produce a longer nucleic acid that is a complimentary copy of the template. A primer may be DNA, RNA, an analog thereof (i.e., an artificial nucleic acid), or any combination thereof. A primer may have any suitable length, such as at least about 10, 15, 20, or 30 nucleotides. Exemplary primers are synthesized chemically. Primers may be supplied as at least one pair of primers for amplification of at least one nucleic acid target. A pair of primers may be a sense primer and an antisense primer that collectively define the opposing ends (and thus the length) of a resulting amplicon.

A probe can be a nucleic acid connected to at least one label, such as at least one dye. A probe may be a sequence specific binding partner for a nucleic acid target and/or amplicon. The probe may be designed to enable detection of target amplification based on fluorescence resonance energy transfer (FRET). An exemplary probe for the nucleic acid assays disclosed herein includes one or more nucleic acids connected to a pair of dyes that collectively exhibit fluorescence resonance energy transfer (FRET) when proximate one another. The pair of dyes may provide first and second emitters, or an emitter and a quencher, among others. Fluorescence emission from the pair of dyes changes when the dyes are separated from one another, such as by cleavage of the probe during primer extension (e.g., a 5′ nuclease assay, such as with a TAQMAN probe), or when the probe hybridizes to an amplicon (e.g., a molecular beacon probe). The nucleic acid portion of the probe may have any suitable structure or origin, for example, the portion may be a locked nucleic acid, a member of a universal probe library, or the like. In other cases, a probe and one of the primers of a primer pair may be combined in the same molecule (e.g., Amplifluor® primers or Scorpion® primers). As an example, the primer-probe molecule may include a primer sequence at its 3′ end and a molecular beacon-style probe at its 5′ end. With this arrangement, related primer-probe molecules labeled with different dyes can be used in a multiplexed assay with the same reverse primer to quantify target sequences differing by a single nucleotide (single nucleotide polymorphisms (SNPs)). Another exemplary probe for droplet-based nucleic acid assays is a Plexor® primer.

A label can be an identifying and/or distinguishing marker or identifier connected to or incorporated into any entity, such as a compound, biological particle (e.g., a cell, bacteria, spore, virus, or organelle), or droplet. A label may, for example, be a dye that renders an entity optically detectable and/or optically distinguishable. Exemplary dyes used for labeling are fluorescent dyes (fluorophores) and fluorescence quenchers. Exemplary fluorescent dyes that can used with the present system include a fluorescent derivative, such as carboxyfluorescein (FAM), and a Pulsar® 650 dye (a derivative of Ru(bpy)₃). FAM has a relatively small Stokes shift, while Pulsar® 650 dye has a very large Stokes shift. Both FAM and Pulsar® 650 dye can be excited with light having a wavelength of approximately 460-480 nm. FAM emits light with a maximum wavelength of about 520 nm (with no substantial emission at 650 nm), while Pulsar® 650 dye emits light with a maximum wavelength of about 650 nm (with no substantial emission at 520 nm). Carboxyfluorescein can be paired in a probe with, for example, BLACK HOLE Quencher™ 1 dye, and Pulsar® 650 dye can be paired in a probe with, for example, BLACK HOLE Quencher™ 2 dye. For example, fluorescent dyes include, but are not limited to, DAPI, 5-FAM, 6-FAM, 5(6)-FAM, 5-ROX, 6-ROX, 5,6-ROX, 5-TAMRA, 6-TAMRA, 5(6)-TAMRA SYBR, TET, JOE, VIC, HEX, R6G, Cy3, NED, Cy3.5, Texas Red, Cy5, and Cy5.5.

A reporter can be a compound or set of compounds that reports a condition, such as the extent of a reaction. Exemplary reporters comprise at least one dye, such as a fluorescent dye or an energy transfer pair, and/or at least one oligonucleotide. Exemplary reporters for nucleic acid amplification assays may include a probe and/or an intercalating dye (e.g., SYBR Green, ethidium bromide, etc.).

A binding partner can be a member of a pair of members that bind to one another. Each member may be a compound or biological particle (e.g., a cell, bacteria, spore, virus, organelle, or the like), among others. Binding partners may bind specifically to one another. Specific binding may be characterized by a dissociation constant of less than about 10⁻⁴, 10⁻⁶, 10⁻⁸, or 10⁻¹⁰ M. Exemplary specific binding partners include biotin and avidin/streptavidin, a sense nucleic acid and a complementary antisense nucleic acid (e.g., a probe and an amplicon), a primer and its target, an antibody and a corresponding antigen, a receptor and its ligand, and the like.

The systems in the present disclosure may provide diagnosis of a genetic disease by testing for a presence (or absence for diseases characterized by deletions) of a nucleic acid target for the genetic disease. Illustrative genetic diseases that may be diagnosed with suitable disease-specific primers include sickle cell anemia, cystic fibrosis (CF), Prader-Willi syndrome (PWS), beta-thalassemia, prothrombin thrombophilia, Williams syndrome, Angelman syndrome, fragile X syndrome, Factor V Leiden, or the like. Exemplary primers include hemoglobin sequences for sickle cell anemia, cystic fibrosis transmembrane conductance regulator (CFTR) gene sequences for cystic fibrosis, and so on. The diagnosis may include determining the variant for diseases having more than one form (e.g., distinguishing among sickle trait (AS), sickle cell anemia (SS), hemoglobin SC disease, hemoglobin SD disease, and hemoglobin SO disease, among others, for hemoglobin-related diseases). These tests may be performed pre- or post-natally, to screen for a single disease or variant, or for a panel of diseases and/or variants (for example, in prenatal screens, using genetic material obtained from an amniocentesis or maternal peripheral circulation, among others).

The systems in the present disclosure may provide detection and/or delineation of native and/or pathogenic gene transcripts. For example, primers may be chosen to amplify one or more targets that signal initiation and/or amplification of any pathophysiological messaging cascade (e.g., TNF-alpha, one or more interleukins, NF-kappaB, one or more inflammatory modulators/mediators), viable infectious agent proliferation, etc.

The systems in the present disclosure may be utilized (e.g., forensically) to determine identity, paternity, maternity, sibling relationships, twin typing, genealogy, etc. These tests may be performed by amplifying nucleic acid from the individuals at issue (including self for identity testing) and comparing nucleic acid sequences, nucleic acid restriction patterns, etc. Suitable nucleic acids may include Y-chromosome DNA for paternity testing, mitochondria DNA for maternity testing, genomic DNA for sibling tests, etc.

The systems in the present disclosure may provide detection of viruses, their transcripts, their drug sensitivity, and/or pathogenic consequences thereof. For example, the tests may use primers that amplify one or more viral targets (e.g., at least a region of one or more viral genes or transcripts), to diagnose and/or monitor viral infections, measure viral loads, genotype and/or serotype viruses, and/or the like. Exemplary viral targets may include and/or may be provided by, but are not limited to, hepatitis C virus (HCV), hepatitis B virus (HPB), human papilloma virus (HPV), human immunodeficiency virus (HIV), cytomegalovirus (CMV), Epstein-Barr virus (EBV), respiratory syncytial virus (RSV), West Nile virus (WNV), varicella zoster virus (VZV), parvovirus, rubella virus, alphavirus, adenovirus, coxsackievirus, human T-lymphotropic virus 1 (HTLV-1), herpes virus (including for Kaposi's sarcoma), influenza virus, enterovirus, and/or the like. In some embodiments, the tests may provide detection/identification of new viral pathogens.

The systems in the present disclosure may provide detection of prokaryotic organisms (i.e., bacteria), their transcripts, their drug sensitivity, and/or pathogenic consequences thereof (e.g., bacterial infections). For example, the tests may use primers that amplify one or more bacterial targets (e.g., at least a region of one or more bacterial genes or transcripts). Suitable bacteria that may be detected include, but are not limited to, gram-positive bacteria, gram-negative bacteria, and/or other fastidious infectious agents. Exemplary bacterial diseases/conditions that may be diagnosed and/or monitored include sexually transmitted diseases (e.g., gonorrhea (GC), Chlamydia (CT), syphilis, etc.); healthcare associated infections (HAIs), such as methicillin-resistant Staphylococcus aureus (MRSA), Clostridium difficile (C. diff.), vancomycin resistant entereococci (VRE), etc.; Group B streptococcus (GBS); mycobacteria (e.g., causing tuberculosis, leprosy, etc.); and/or the like.

The tests in the present disclosure may provide detection of fungi (single-celled (e.g., yeast) and/or multi-celled), their transcripts, pathogenic consequences thereof (e.g., fungal infections), and/or drug sensitivity. For example, the tests may use primers that amplify one or more fungal targets (e.g., at least a region of one or more viral genes or transcripts). Exemplary types of fungal infections that may be diagnosed and/or monitored may be caused by Histoplasma (e.g., causing histoplasmosis), Blastomyces (e.g., causing blastomycosis), Cryptococcus (e.g., causing meningitis), Coccidia (e.g., causing diarrhea), Candida, Sporothrix genuses of fungi, and/or the like.

The tests in the present disclosure may be used for screening, diagnosis, monitoring, and/or designing treatment of diseases such as cancer. For example, tests for cancer may detect one or more cancer mutations (e.g., her2/neu, BRACA-1, etc.), insertion/deletion/fusion genes (bcr-abl, k-ras, EFGR, etc.), amplified genes, epigenetic modifications, etc.; may identify cancer stem cells; may identify, monitor, and/or evaluate residual cancer disease burden, p53 margin assessment, etc.; and/or the like. These tests may use any suitable cancer markers as targets and may be applied to any suitable type of cancer, such as bladder cancer, bone cancer, breast cancer, brain cancer, cervical cancer, colorectal cancer, esophageal cancer, gastric cancer, oropharyngeal cancer, ovarian cancer, prostate cancer, uterine cancer, leukemia, lymphoma, myeloma, melanoma, etc.

### Tunability and Waste Management

The present disclosure provides compositions and methods for managing waste for droplet-based assays. The oil-immiscible fluid used for spacing, diluting, focusing, or detecting may be aqueous or air. When the oil-immiscible fluid is air, there is no additional waste added to the detection system, providing an advantage over the oil-based dilution or spacing fluid. Alternatively, the oil-immiscible fluid may be an aqueous fluid; for example, it may primarily be made up of water.

The amount of oil waste generated in the systems described herein can be greater than or equal to about 1-, 2-, 3-, 4-, 5-, 6-, 7-, 8-, 9-, 10-, 20-, 30-, 40-, 50-, 75-, 100-, 500-, 1000-, or 1500-fold less than the amount of waste generated by the same system that uses a non-aqueous fluid for spacing, diluting, focusing, and/or detecting droplets.

The aqueous fluids provided herein also may have tunable properties, such as viscosity, surface tension, antimicrobial property, etc. The tunable properties may be adjusted by the addition of at least one additive, for example, surfactant, viscosity-enhancing agent, or antimicrobial agent. In some cases, the viscosity of an aqueous fluid with an additive is at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.2, 3.4, 3.6, 3.8, 4.0, 4.5, 5.0, 5.5, 6.0, 7.0, 8.0, 9.0, 10, 15, 20, 25, 30, 35, 40, 45, 50, 55, 60, 65, 70, 75, 80, 85, 90, 100, 150, 200, 250, 300, 350, 400, 450, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 400,000, 500,000, 600,000, 700,000, 800,000, or even more times the viscosity of the aqueous fluid without the additive. In some cases, the surface tension of an aqueous fluid with an additive is at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.2, 3.4, 3.6, 3.8, 4.0, 4.5, 5.0, 5.5, 6.0, 7.0, 8.0, 9.0, 10, 15, 20, 25, 30, 35, 40, 45, 50, 55, 60, 65, 70, 75, 80, 85, 90, 100, 150, 200, 250, 300, 350, 400, 450, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 400,000, 500,000, 600,000, 700,000, 800,000, or even more times the surface tension of the aqueous fluid without the additive. In some cases, the amount of microbial and/or fungi in waste with an additive is no more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 38%, 40%, 45%, 50%, 60%, 70%, or 80% of the amount of microbial and/or fungi present in the waste without the additive.

Furthermore, the addition of antimicrobial agent to an oil-immiscible fluid as described herein may suppress the growth of bacteria and fungi in the waste and/or reduce certain risks associated with waste management, providing a safer working environment.

### Computer Systems

Provided herein are computer systems for implementing methods of the present disclosure, such as droplet formation, spacing and droplet detection. Computer systems of the present disclosure can control or regulate various aspects of droplet formation, spacing and droplet detection, such as regulating the source of positive pressure or negative pressure (vacuum) to regulate fluid flow, regulating a droplet detector in communication with a computer system, collecting and storing data, and aiding in data analysis.

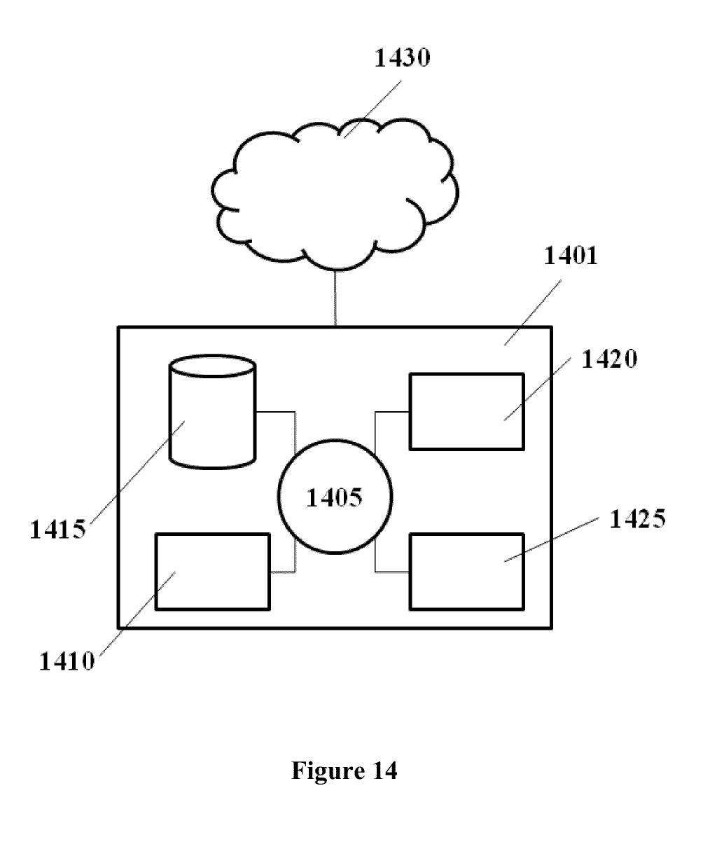

FIG. 14 shows a computer system 1401 that is programmed or otherwise configured to regulate droplet formation, spacing and droplet detection. The computer system 1401 can be separate from a droplet generator but in communication with the droplet generator, or be part of the droplet generator, such as integrated with the droplet generator. The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. Examples of operations performed by the CPU 1405 can include fetch, decode, execute, and writeback.

The computer system 1401 can communicate with one or more remote computer systems through the network 1430. For instance, the computer system 1401 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1401 via the network 1430.

Methods as described herein can be implemented by way of machine (e.g., computer processor) executable code stored on an electronic storage location of the computer system 1401, such as, for example, on the memory 1410 or electronic storage unit 1415. The machine executable or machine readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### EXAMPLES

The examples below are illustrative of various embodiments of the present disclosure and are not intended to be limiting.

### Example 1

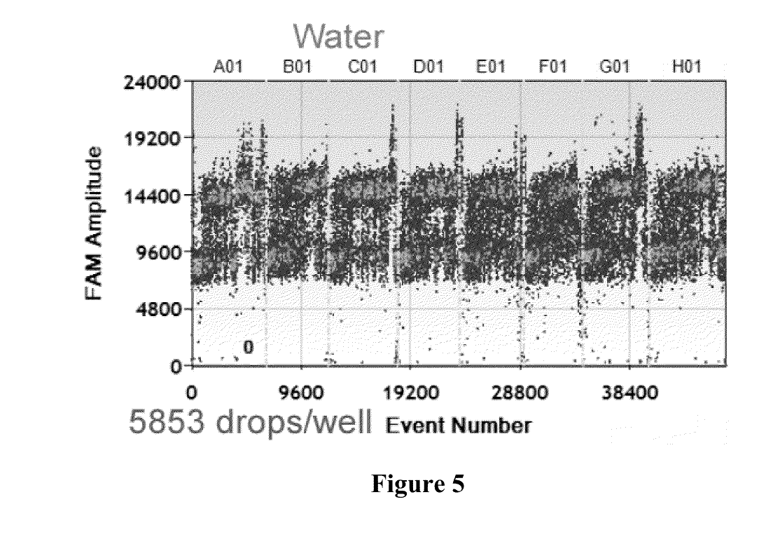

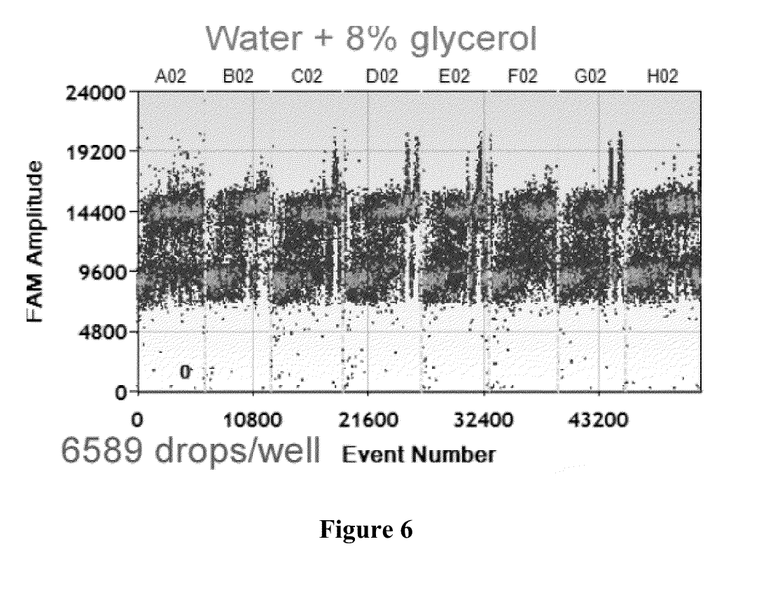

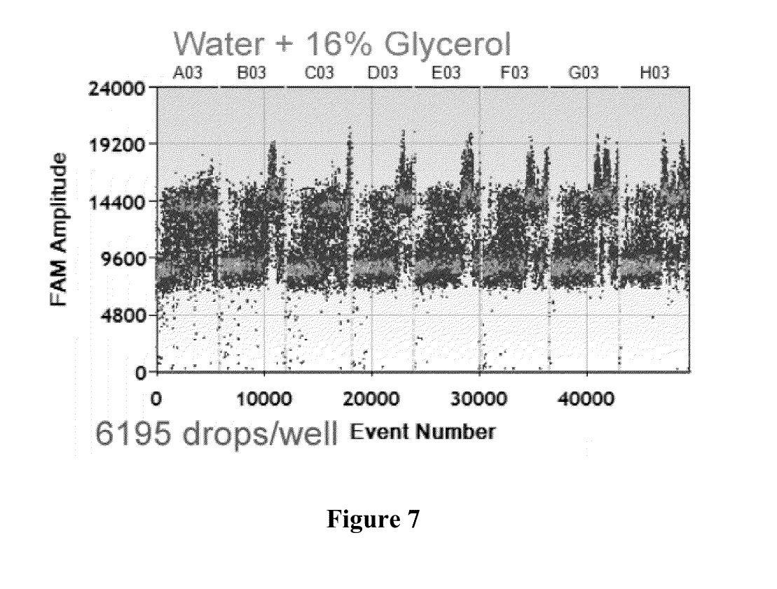

Droplets are generated using a droplet generator, such as a droplet generator described in U.S. Patent Publication No. 2010/0173394, which is entirely incorporated herein by reference. The droplets are formed using droplet generation oil and a PCR mixture comprising DNA polymerase and primers. The droplets in this example are prepared to include a dye, but they do not include a DNA sample. The droplets are then thermally cycled. Next, the droplets are directed to a droplet spacing and/or focusing device, such as the device of FIG. 3, which includes a droplet reader. In the device, the droplets are subjected to flow using an oil-immiscible carrier fluid. The droplets are detected using the droplet reader. FIG. 5-FIG. 7 shows that non-optimal oil-immiscible fluids (FIG. 5 for water, FIG. 6 for water+8% glycerol, and FIG. 7 for water+16% glycerol) lead to reduced droplet counts as well as noisy data. This is illustrated by the spread of the carboxyfluorescein (FAM) amplitude.

### Example 2

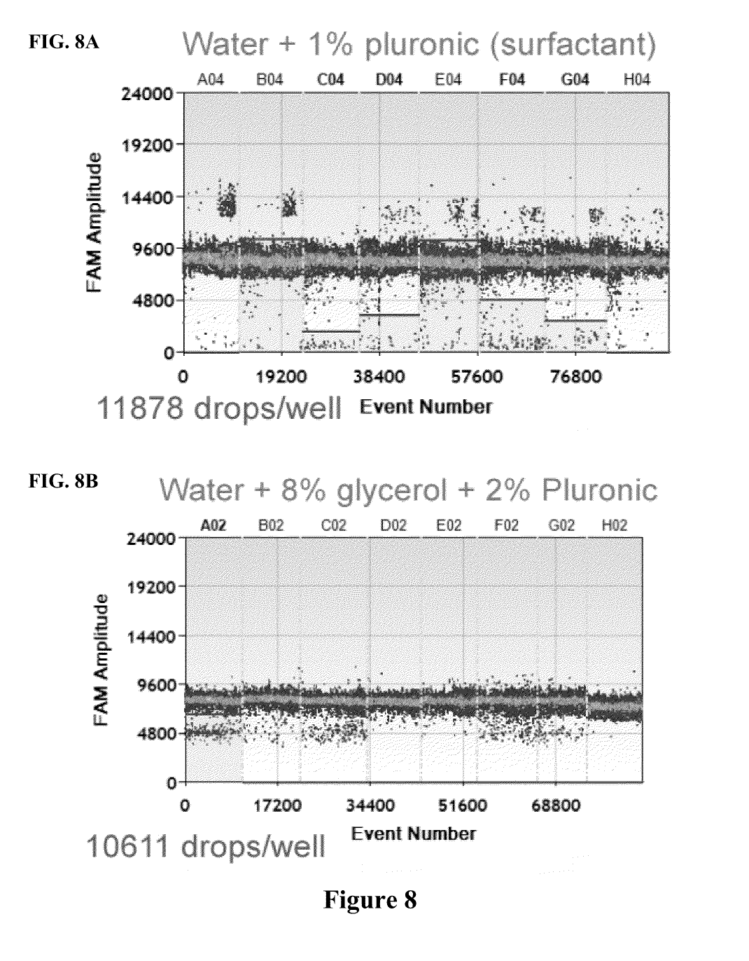

In another example, droplets are generated in the manner described in Example 1. The droplets in this example are prepared to include a dye, but they do not include a DNA sample. The droplets are then thermally cycled. Next, the droplets are directed to a droplet spacing and/or focusing device, such as the device of FIG. 3, which includes a droplet reader. In the device, the droplets are subjected to flow using an aqueous carrier fluid. The droplets are detected using the droplet reader. Droplet detection is optimized by optimizing selected properties of the aqueous carrier fluid. FIG. 8 shows that optimization of aqueous carrier fluid properties can increase droplet counts and improve data quality. FIG. 8A depicts the graph with 1% Pluronic® as additive to water. FIG. 8B depicts the graph with 8% glycerol and 2% Pluronic® to water.

### Example 3

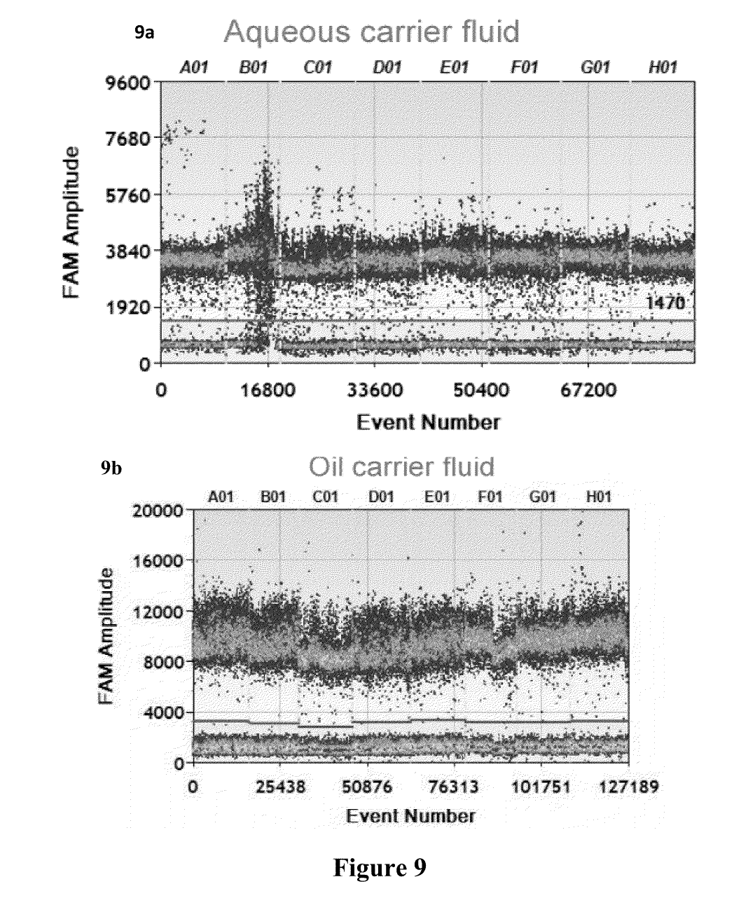

In another example, droplets are generated in the manner set forth in Example 1. The droplets in this example are prepared to include a dye and a DNA sample. The droplets are then thermally cycled to induce DNA amplification. Next, the droplets are directed to a droplet spacing and/or focusing device, such as the device of FIG. 3, which includes a droplet reader. In one experiment, the droplets are subjected to flow using an aqueous carrier fluid. A comparable experiment is conducted by flowing droplets using an oil carrier fluid. In both experiments, the droplets are detected using the droplet reader. FIG. 9 shows that biological assay data quality of the aqueous carrier fluid is comparable to that of the oil carrier fluid. The upper panel of FIG. 9 is for the aqueous carrier fluid, and the lower panel of FIG. 9 is for the oil carrier fluid.

### Example 4

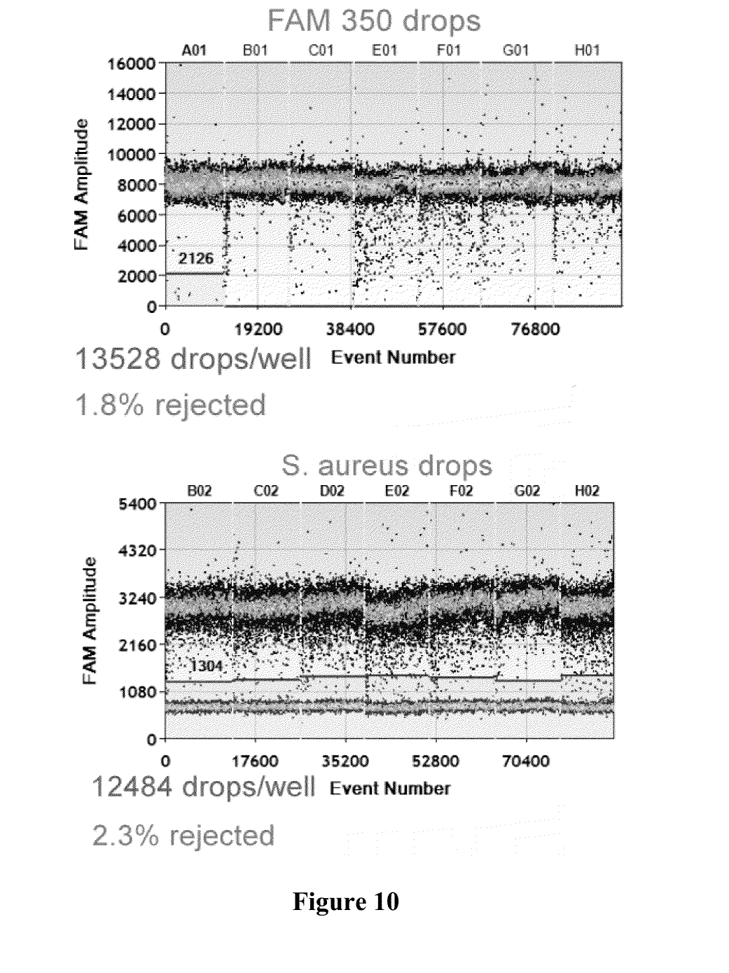

In another example, droplets are generated in the manner set forth in Example 1. The droplets are then thermally cycled. Next, the droplets are directed to a droplet spacing and/or focusing device, such as the device of FIG. 3, which includes a droplet reader. In the device, the droplets are subjected to flow using an aqueous carrier fluid. The droplets are detected using the droplet reader. FIG. 10 shows that higher singulation ratio can decrease rejected droplets. The droplets in FIG. 10 (top panel) are prepared to include a dye, but they do not include a DNA sample. The droplets in FIG. 10 (bottom panel) are prepared to include a DNA sample and a dye. The droplets in the bottom panel are thermally cycled to induce DNA amplification.

### Example 5

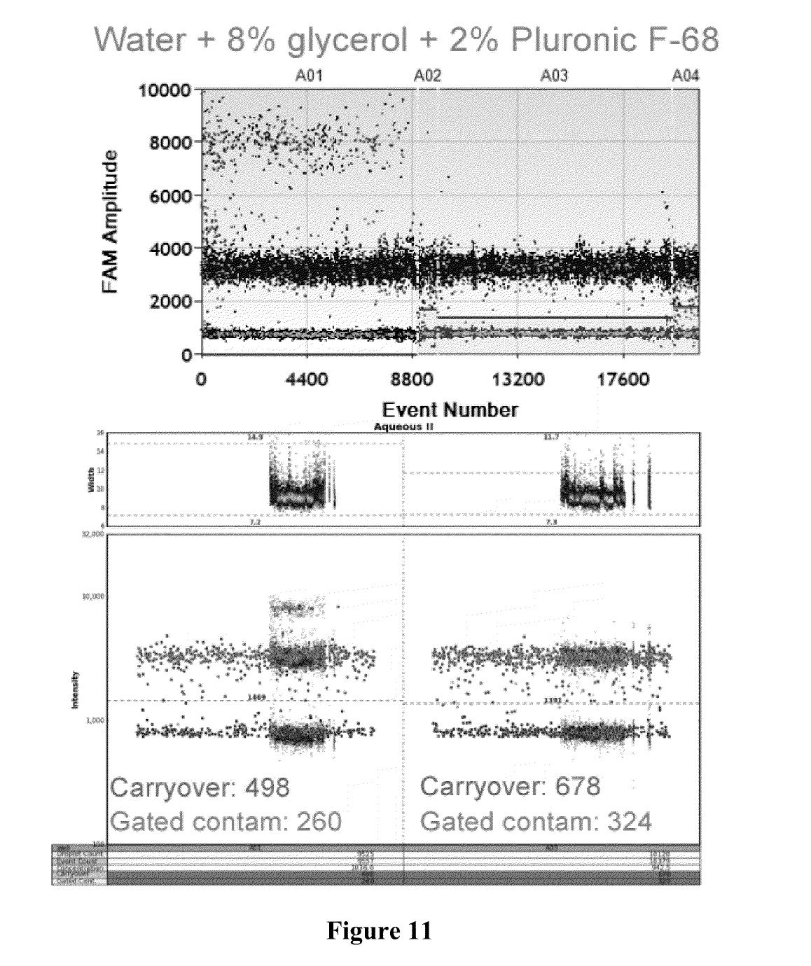

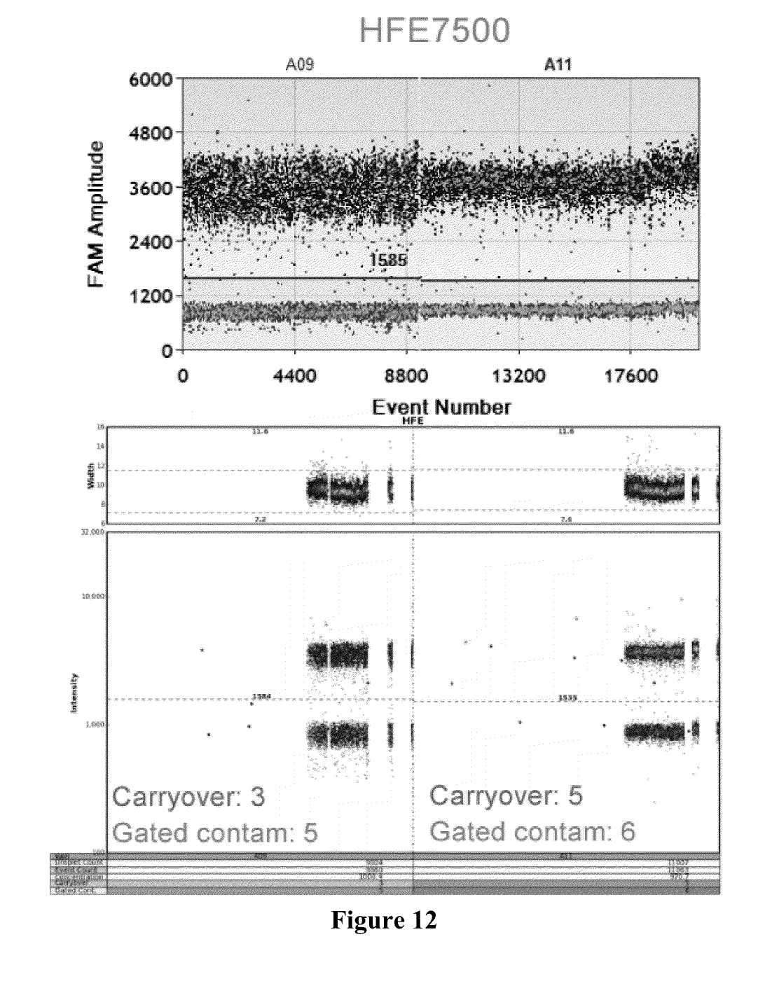

In another example, droplets are generated in the manner set forth in Example 1, with the exception that the droplets are prepared to include a DNA sample. The droplets are thermally cycled and subjected to flow using an aqueous carrier fluid. FIG. 11 is a graphical representation of the fluorescence amplitudes of droplets detected after the droplets are contacted with an oil-immiscible fluid comprising water, 8% glycerol, and 2% Pluronic® F-68 surfactant. FIG. 12 is a graphical representation of the fluorescence amplitudes of droplets detected after the droplets are contacted with a focusing fluid comprising HFE-7500 oil. FIG. 11 shows that carryover can be relatively high with aqueous dilution fluid (and no tip wiping), and FIG. 12 shows that the carryover can be relative low with a focusing fluid comprising oil. The droplets in FIGS. 11 and 12 are prepared to include a DNA sample and a dye, and they are thermally cycled to induce DNA amplification.

### Example 6

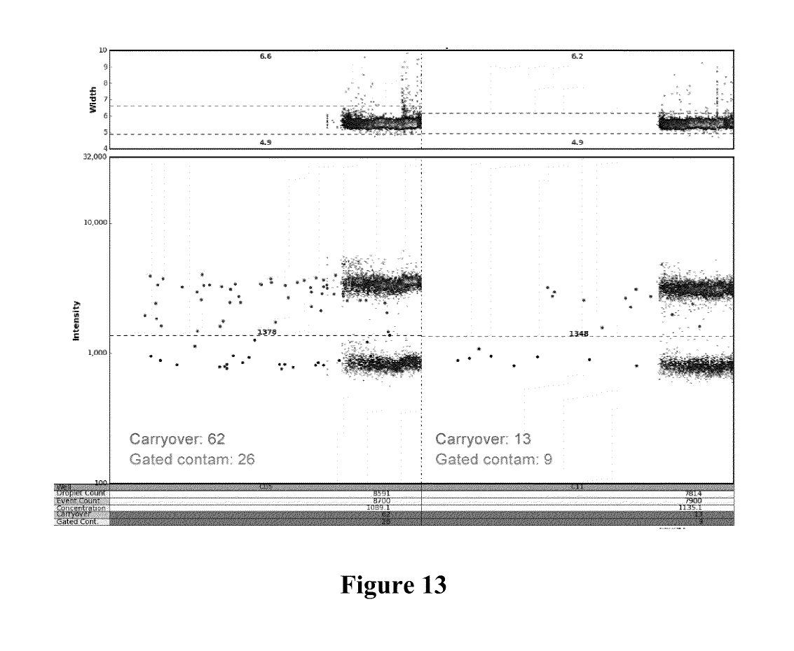

In another example, droplets are generated in the manner set forth in Example 1 and stored in a 96-well plate. The droplets are prepared to include a DNA sample. The droplets are then thermally cycled to induce nucleic acid amplification. Next, the droplets are directed to a droplet spacing and/or focusing device, such as the device of FIG. 3, which includes a droplet reader. The droplets are directed to the device using a pick-up tip (sipper) of the droplet reader, which punctures a foil of the 96-well plate, picks up the droplets in individual wells using suction (negative pressure), and flows the droplets through the device and in sensing proximity to the droplet reader. The tip can be wiped between pickups. The tip can be wiped using an oil wash reservoir containing a wash solution, which can include an oxidizing agent (e.g., bleach), or by physically wiping the tip (e.g., with a lab tissue). FIG. 13 shows that carryover can be dramatically reduced by wiping outside of the tip. The left and right panels of FIG. 13 are for a tip that has been wiped. For comparison, FIG. 11 (lower panel) is for an unwiped tip. FIG. 13 shows that wiping can substantially reduce carryover. Tip cleaning (e.g., wiping) can be further optimized to achieve carryover levels shown in the lower panel of FIG. 12. The droplets in FIGS. 11-13 are prepared to include a DNA sample and a dye, and they are thermally cycled to induce DNA amplification.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A system for detecting droplets, comprising:
a. a detector device comprising an input flow path, an intersection region, and an output flow path, wherein the intersection region is downstream of the input flow path and the output flow path is downstream of the intersection region;
b. droplets located within the input flow path; and
c. an aqueous fluid for separating the droplets wherein the droplets are introduced to the aqueous fluid at the intersection region.

2. The system of claim 1, wherein the input flow path comprises a continuous phase of non-aqueous fluid.

3. The system of claim 2, wherein the non-aqueous fluid is an aqueous-immiscible fluid.

4. The system of claim 2, wherein the non-aqueous fluid is an oil.

5. The system of claim 1, wherein the output flow path comprises a continuous phase of aqueous fluid.

6. The system of claim 5, wherein the aqueous fluid comprises a surfactant.

7. The system of claim 1, wherein the droplets in the output flow path have an inner core containing an aqueous fluid that is encapsulated with a non-aqueous fluid.

8. The system of claim 7, wherein the non-aqueous fluid is a continuous phase.

9. The system of claim 7, wherein the non-aqueous fluid is a discontinuous phase.

10. A system for detecting droplets, comprising:
a. a detector device comprising an input flow path, an intersection region, and an output flow path, wherein the intersection region is downstream of the input flow path and the output flow path is downstream of the intersection region; and
b. an oil-immiscible fluid for separating the droplets, wherein the oil-immiscible fluid is introduced to the droplets at the intersection region;
wherein the continuous phase of fluid within the input flow path is a non-aqueous fluid and wherein the inner surface of the output flow path is coated with the non-aqueous fluid with a thickness that is at least 0.01% of the diameter of the outflow path, thereby narrowing the aperture of the output flow path; and
wherein the droplets in the output flow path have an inner core containing an aqueous fluid that is encapsulated with the non-aqueous fluid.

11. The system of claim 10, wherein the thickness is at least 0.1% of the diameter of the output flow path.

12. The system of claim 10, wherein the thickness is at least 1% of the diameter of the output flow path.

13. The system of claim 10, wherein the thickness is at least 5% of the diameter of the output flow path.

14. The system of claim 10, wherein the thickness is in a range of 1%-90% of the diameter of the output flow path.

15-17. (canceled)

18. The system of claim 1, wherein the droplets are emulsified droplets.

19-27. (canceled)

28. A method of separating droplets, comprising:
(a) flowing a stream of non-aqueous fluid comprising the droplets along a flow path comprising: (i) an input flow path, (ii) an intersection region, and (iii) a downstream output flow path; and
(b) introducing a stream of oil-immiscible fluid to the intersection region;
wherein the average distance between the droplets in the output flow path is greater than the average distance between the droplets within the input flow path.

29. (canceled)

30. (canceled)

31. The method of claim 28, wherein the output flow path comprises: (a) a continuous phase of oil-immiscible fluid; and (b) aqueous droplets encapsulated by a layer of non-aqueous fluid.

32. The method of claim 28, wherein the flow paths of the non-aqueous fluid and that of the oil-immiscible fluid are substantially perpendicular.

33. The method of claim 28, wherein the oil-immiscible fluid comprises water.

34. The method of claim 28, wherein the oil-immiscible fluid comprises air.

35. The method of claim 33, wherein the water comprises a surfactant.

36-73. (canceled)

74. The system of claim 10, wherein the droplets are emulsified droplets.
